(19) United States
(12) Patent Application Publication
(10) Pub. No.: US 2012/0149631 A1
(43) Pub. Date: Jun. 14, 2012

(54) ANTIMICROBIAL AND ANTIBIOFILM ACTIVITY OF CATHELICIDINS

(75) Inventors: Frank Delatour, Fairfax, VA (US); Monique Van Hoek, Centerville, VA (US); Barney Bishop, Annandale, VA (US)

(73) Assignee: George Mason Intellectual Properties, Inc

(21) Appl. No.: 13/378,638

(22) PCT Filed: Jun. 16, 2010
(86) PCT No.: PCT/US10/38810

§ 371 Date: Feb. 29, 2012

Related U.S. Application Data
(62) Division of application No. 61/187,365, filed on Jun. 16, 2009.

(51) Int. Cl. A01N 37/18; A01P 1/00; C12N 15/63; C07K 7/06; C07K 2/00

(52) U.S. Cl. 514/2.8; 530/327; 530/300; 435/320.1

(57) ABSTRACT

The present disclosure relates to peptides, and fragments thereof, conferring antimicrobial and/or antibiofilm growth, as well as products and methodology for using same.

### CROSS-REFERENCE TO RELATED APPLICATION

This application claims priority to U.S. Provisional Application No. 61/187,365, filed Jun. 16, 2009, the disclosure of which is herein incorporated by reference.

### FIELD OF TECHNOLOGY

The present disclosure relates to peptides conferring antimicrobial and/or antibiofilm growth. Included are cathelicidins, such as helical cathelicidins and fragments thereof, from a variety of species.

### INTRODUCTION

Multicellular organisms, such as humans, are constantly exposed to many different types of pathogenic microorganisms. Infection by these microbes is generally fended off by a variety of responses produced by the innate and adaptive immune system. One such response of the innate immune system is the release and subsequent effects of antimicrobial peptides (AMP). These small amino acid chains are generally produced in response to invasion by bacteria, fungi, viruses and protozoa.

Typically, antimicrobial peptides are short, about 12 to 100 amino acids in length, and possess a positive charge which can differ greatly depending upon the length and the amino acid composition of the peptide (33). They have evolved over thousands of years into effective defensive weapons against the previously mentioned organisms and are found everywhere from single celled microorganisms to extremely complex ones such as humans (32, 33). The expression of these peptides can be either constitutive or inducible, and the fact that hundreds of such peptides have been identified emphasizes their importance to the innate immune system in a wide range of organisms. The peptides possess not only the ability to directly kill invaders, but also the ability to stimulate effector molecules of the host immune system.

It has been noted that mammalian AMP's can neutralize the septic effects of bacterial lipopolysaccharide (LPS), induce wound repair, and act as a chemoattractant for monocytes and T cells of the adaptive immune system (32, 33). Though many induce similar effects, these molecules are known to share little homology when it comes to their amino acid sequence. However, their most well known and well studied attribute is quite simple yet extremely effective: they specifically target and disrupt the cellular membrane of microorganisms, and to date, there have been very few instances of pathogens developing resistance to AMPs.

Almost all AMPs are amphipathic in design with different regions of hydrophobic and cationinc amino acids that are found in different places of the molecule. Typically, AMPs are derived from larger precursors which originally contained a signal sequence. Glycosylation, proteolytic cleavage, amidation of the carboxyl terminal as well as halogenation are all post translational modifications which are known to occur to the precursors of the AMPs (32). After proper processing, these molecules generally adopt four major types of structure: amphiplilic peptides with two to four β-strands, amphipathic α-helices, loop structures, and extended structures (32-34). Once the appropriate modifications have taken place, the mature AMPs have the ability to associate with the outermost leaflet of bacterial membranes.

The antibacterial properties of some of the first identified AMP's are well studied. Regardless of their final target and mechanism of action, these peptides must at some point interact with the membrane of the target bacteria, specifically the outermost leaflet. This outermost leaflet of bacterial membranes is comprised of a large number of lipids that have negatively charged phospholipid head groups which is in direct contrast to the outer leaflet of the membranes of plants and animals which are made up of lipids that have no charge on their head groups (32). With respect to plants and animals, the negatively charged head groups are found mainly on the inner leaflet which faces the cytoplasm.

### SUMMARY

In one aspect, there is provided an isolated cathelicidin conferring antimicrobial activity against Aggregatibacter actinomycetemcomitans, wherein said cathelicidin is K9CATH, BMAP-28, ATRA-1, ATRA-2, ATRA-1A, ATRA-1P, or PMAP-37.

In one aspect, there is provided an isolated peptide conferring antimicrobial activity against a gram-negative bacterium wherein said peptide is K9CATH, BMAP-28, ATRA-1, ATRA-2, ATRA-1A, ATRA-1P, or PMAP-37. In one embodiment, the gram-negative bacterium is A. actinomycetemcomitans, F. tularensis, or E. coli.

In another aspect, there is provided an isolated peptide consisting essentially of ATRA-1, ATRA-2, ATRA-1A, or ATRA-1P.

In another aspect, there is provided a product comprising at least one of ATRA-1, ATRA-2, ATRA-1A, and ATRA-1P. In one embodiment, said product is a mouthwash, toothpaste, antibacterial gel, soap, detergent, antimicrobial product, or antibiofilm product.

In another aspect, there is provided a vector comprising a sequence encoding at least one of ATRA-1, ATRA-2, ATRA-1A, and ATRA-1P.

In another aspect, there is provided an isolated cathelicidin conferring antibiofilm activity against F. novicida, wherein said cathelicidin is LL-37, ATRA-1, ATRA-2, ATRA-1A, or ATRA-1P.

In another aspect, there is provided an isolated cathelicidin conferring antimicrobial activity against F. novicida, wherein said cathelicidin is ATRA-1 or ATRA-2. In one embodiment, said cathelicidin is ATRA-1.

In another aspect, there is a method for sterilizing a surface against a gram-negative bacterium, comprising contacting said surface with at least one of K9CATH, BMAP-28, ATRA-1, ATRA-2, ATRA-1A, ATRA-1P, or PMAP-37. In one embodiment, said gram-negative bacterium is A. actinomycetemcomitans, F. tularensis, or E. coli.

In another aspect, there is provided a method for inhibiting growth of A. actinomycetemcomitans, comprising exposing a surface or organism or product to at least one of ATRA-1, ATRA-2, ATRA-1A, and ATRA-1P.

In another aspect, there is provided a method for inhibiting growth of F. tularensis, comprising exposing a surface or organism or product to at least one of ATRA-1, ATRA-2, ATRA-1A, and ATRA-1P.

In another aspect, there is provided a mouthwash comprising at least one of ATRA-1, ATRA-2, ATRA-1A, and ATRA-1P.

### BRIEF DESCRIPTION OF THE DRAWINGS

FIG. 1: Susceptibility of A. actinomycetemcomitans Y4 to various concentrations of CAP-18. Inhibition of growth calculated via enumeration of CFU's after 3 hour incubation with peptide.

FIG. 2: Susceptibility of A. actinomycetemcomitans Y4 to various concentrations of K9CATH. Inhibition of growth calculated via enumeration of CFU's after 3 hour incubation with peptide.

FIG. 3: Susceptibility of A. actinomycetemcomitans Y4 to various concentrations of BMAP-28. Inhibition of growth calculated via enumeration of CFU's after 3 hour incubation with peptide.

FIG. 4: Susceptibility of A. actinomycetemcomitans Y4 to various concentrations of SMAP-29. Inhibition of growth calculated via enumeration of CFU's after 3 hour incubation with peptide.

FIG. 5: Susceptibility of A. actinomycetemcomitans Y4 to various concentrations of PMAP-37. Inhibition of growth calculated via enumeration of CFU's after 3 hour incubation with peptide.

FIG. 6: Susceptibility of A. actinomycetemcomitans Y4 to various concentrations of LL-37. Inhibition of growth calculated via enumeration of CFU's after 3 hour incubation with peptide.

FIG. 7: Susceptibility of A. actinomycetemcomitans Y4 to various concentrations of LL-37 Pentamide. Inhibition of growth calculated via enumeration of CFU's after 3 hour incubation with peptide.

FIG. 8: A. Susceptibility of A. actinomycetemcomitans Y4 to various concentrations of Snake Peptide (Full length). Inhibition of growth calculated via enumeration of CFU's after 3 hour incubation with peptide. B. Susceptibility of E. coli to various concentrations of NA-CATH Peptide (Full length). The EC50 was found to be 0.192 μg/ml (0.0613-0.132), with an R² of 0.990 and a Hill Slope of 1.83 (1.165-2.50).

FIG. 9: Antimicrobial Activity of ATRA-1 Peptide.

A. Susceptibility of A. actinomycetemcomitans Y4 to various concentrations of Snake Peptide 1 (ATRA 1 Fragment). Inhibition of growth calculated via enumeration of CFU's after 3 hour incubation with peptide. B. Susceptibility of E. coli to various concentrations of ATRA-1 Peptide. The EC50 was calculated to be 0.881 μg/ml (0.539-1.44), with an R² of 0.975, and a Hill Slope of 0.683 (0.4824-0.8830).

FIG. 10: Antimicrobial Activity of ATRA-2 Peptide.

A Susceptibility of A. actinomycetemcomitans Y4 to various concentrations of Snake Peptide 2 (ATRA2 Fragment). Inhibition of growth calculated via enumeration of CFU's after 3 hour incubation with peptide. B. Susceptibility of E. coli to various concentrations of ATRA-2. The EC50 was found to be 22.2 μg/ml (14.24-34.60) with an R² of 0.975 and a Hill Slope of 0.835 (0.595 to 1.08).

FIG. 11: Antimicrobial Activity of ATRA-1A Peptide.

A. Susceptibility of A. actinomycetemcomitans to various concentrations of ATRA-1A. Inhibition of growth calculated via enumeration of CFU's after 3 hr incubation with peptide.

B. Susceptibility of E. coli to various concentrations of ATRA-1A. The EC50 for E. coli was calculated to be 0.939 μg/ml (0.726-1.196) with an R² of 0.986. In addition, the Hill Slope was 1.04 (0.760-1.32).

FIG. 12: Antimicrobial Activity of ATRA-1P Peptide.

A. Susceptibility of A. actinomycetemcomitans to various concentrations of ATRA-1P. Inhibition of growth calculated via enumeration of CFU's after 3 hr incubation with peptide.

B. Susceptibility of E. coli to various concentrations of ATRA-Peptide 1P. Inhibition of growth calculated via enumeration of CFU's after 3 hr incubation with peptide. The EC50 for E. coli was found to be 7.05 μg/ml (4.57 to 10.9), with an R² 0.977. The Hill Slope was determined to be 1.27 (−0.391 to 4.78).

FIG. 13: Hemolytic assays with various concentrations of peptides, including full-length NA-CATH Peptide, ATRA-1,2,1A and 1P. Release of heme was measured by absorbance at 540 nm after 1 hour of incubation with peptide.

FIG. 14: CD Spectra of peptides. Minima at 222 nm and 208 nm are hallmarks of helical peptides. The spectra for NA-CATH, ATRA-1 and ATRA-1A in 90 mM SDS are consistent with helical secondary structure. However, the spectra for ATRA-1P and ATRA-2 under similar conditions reflect random coil characteristics.

FIG. 15: Helical wheel projections of ATRA peptides. Helical wheel projections were made using http://kael.org/helical.htm. The sequences of (A) ATRA-1 and (B) ATRA-2 were projected onto the helical backbone.

FIG. 16: Susceptibility of F. novicida to LL-37 and NA-CATH. Inhibition of growth was calculated via enumeration of CFUs after 3 h incubation with various concentrations of the peptide in Buffer Q. The EC50 was found to be 0.24 μg/ml (IC 95% 0.18-0.30, R² 0.988) for LL-37 and 1.54 μg/ml (IC 95% 0.17-1.38, R² 0.983) for NA-CATH.

FIG. 17: Activity of LL-37 and NA-CATH against F. novicida. The peptides were incubated in Buffer Q with F. novicida at the EC50 concentration (0.24 μg/ml for LL-37 and 1.54 μg/ml for NA-CATH). Assays were plated in triplicate at the indicated time points.

FIG. 18: F. novicida biofilm inhibition by LL-37. Biofilm detection on polystyrene (PS) 96 well plate at 37° C. (PS 37° C.) after 48 h of growth in TSB-C is expressed as the absorbance at 570 nm. Growth is indicated in black bars with control set to a 100% and percent biofilm is indicated in grey bars with n=6. This experiment is a representative of three independent trials. * indicates p-value less than 0.01 compared to control.

FIG. 19: LL-37 mRNA expression levels in A549. LL-37 mRNA expression levels were measured using RT-PCR in control (uninfected) and F. novicida infected in serum starved A549 cells (24 h, MOI: 500). LL-37 mRNA expression levels were measured. (*) p-value=0.024 compared to control.

### DETAILED DESCRIPTION

Cathelicidins are a large and diverse group of antimicrobial peptides found in a variety of vertebrate hosts that possess a conserved N-terminal segment, known as the calthelin domain. The C-terminus of the peptide is considered the active portion, and only upon removal of the N-terminus is the peptide considered active. Cathelicidins can be found in their inactive state in the granules of cells of the immune system, but they also occur in the mucosal surfaces of the mouth, lung, and urogenital tract. Because of their diversity, cathelicidins have many different structures and antimicrobial and immunomodulatory properties (36). With the exception of having the conserved N terminus, cathelicidins have been known to possess structures ranging from α-helices to β-hairpin and proline/argenine rich sequences.

The present disclosure provides cathelicidins from a variety of hosts, such as to rabbit, canine, bovine, sheep, reptile, porcine, and human. Such cathelicidins can be used, for example, as antimicrobial agents, antibiofilm agents, as well as in various methods and products, including but not limited to mouthwashes, toothpastes, antibacterial gels, soaps or detergents, as wells as any and all antimicrobial and antibiofilm products.

For example, and in no way limiting, the peptides disclosed herein can be used to protect against A. actinomycetemcomitans, which inhabits microbial biofilms located in the subgingival dental plaque (7). These bacteria can cause an aggressive infection that can quickly lead to rapid loss of the alveolar bone and a disease known as localized aggressive periodontitis (LAP) (8). In addition, colonization by A. actinomycetemcomitans also leads to inflammation of the gingival tissues and destruction of the periodontal ligament (8, 9). A. actinomycetemcomitans has also been implicated in bacterial endocardidits, meningitis, septicemia, tissue abscesses, and osteomyelitis (12-14). A. actinomycetemcomitans has several mechanisms by which it exerts pathogenicity on the host. It has the ability to adhere by pili or by an adhesin, inhabit oral biofilms, and secrete virulence factors such as toxins or immunomodulatory molecules. Additionally, A. actinomycetemcomitans can cause the resorption of bone.

Likewise, the peptides disclosed herein can protect against other microorganisms such as E. coli and Francisella tularensis. Francisella, a gram-negative zoonotic organism that causes the disease tularemia, directly infects the human lung Type II alveolar, and can also form biofilms.

All technical terms used herein are terms commonly used in biochemistry, molecular biology, and microbiology, and can be understood by one of ordinary skill in the art. Those technical terms can be found in: Molecular Cloning: A Laboratory Manual, 3rd ed., vol. 1-3, ed. Sambrook and Russel, Cold Spring Harbor Laboratory Press, Cold Spring Harbor, N.Y., 2001; Current Protocols in Molecular Biology, ed. Ausubel et al., Greene Publishing Associates and Wiley-Interscience, New York, 1988 (with periodic updates); Short Protocols in Molecular Biology: A Compendium of Methods from Current Protocols in Molecular Biology, 5th ed., vol. 1-2, ed. Ausubel et al., John Wiley & Sons, Inc., 2002; Genome Analysis: A Laboratory Manual, vol. 1-2, ed. Green et al., Cold Spring Harbor Laboratory Press, Cold Spring Harbor, N.Y., 1997.

A. actinomycetemcomitans is a Gram-negative bacterium that plays a role in the two most prevalent oral diseases, dental caries and periodontitis. A. actinomycetemcomitans is a facultative anaerobe that grows best in an aerobic environment enriched with carbon dioxide. This species of bacteria is divided into six different serotypes based on the differences in LPS O-antigens (3). For instance, Serotype A contains a deoxy-D-talan component, whereas Serotype B is made up of a rhamnose/fucomse repeating unit (4, 5). Serotype B is recovered more frequently in patients whose symptoms include greater tissue destruction and bone loss, but all serotypes are believed to be pathogenic (6).

Antimicrobial activity means that a peptide destroys and/or prevents the growth or proliferation of a microorganism. For example, and in no way limiting, a cathelicidin peptide may destroy bacterial growth. For a given peptide, for example, antimicrobial activity can be determined as a function of bacterial survival based on the ratio of the number of colonies on the plates corresponding to the peptide concentration and the average number of colonies observed for assay cultures lacking peptide. The peptide concentration required to kill 50% of the viable bacteria in the assay cultures (EC50) can be determined by plotting percent mortality as a function of the log of peptide concentration (log μg/ml) and fitting the data using methods readily known in the art.

Antibiofilm activity: means that a peptide destroys and/or prevents the growth or proliferation of, a biofilm. For example, and in no way limiting, a cathelicidin peptide may destroy bacterial growth in a biofilm or can inhibit the production of biofilm without inhibiting bacterial growth. Antibiofilm activity can be measured as a function of the peptide concentration required to kill 50% of the viable bacteria in the biofilm (EC50).

Cathelicidin refers to a large and diverse collection of cationic antimicrobial peptides found in a variety of vertebrate hosts. Cathelicidins possess a conserved N-terminal segment, known as the calthelin domain, which is removed via proteolytic cleavage in order to form the mature peptide. The C-terminus of the peptide is considered the active portion, and only upon removal of the N-terminus is the peptide considered active. Cathelicidins can be found in their inactive state in the granules of cells of the immune system, but they have also been found in the mucosal surfaces of the mouth, lung, and urogenital tract. Exemplary cathelicidins include but are not limited to rabbit CAP-18, canine K9CATH, bovine BMAP-28, sheep SMAP-29, reptile SNAKE 1 and SNAKE 2, porcine PMAP-37, and human LL-37.

Francisella tularensis is a gram-negative zoonotic organism that causes the disease tularemia, directly infects the human lung, A549 Type II alveolar cells, and can also form biofilms.

Helical Cathelicidin refers to a classification of cathelicidins where a large portion of a cathelicidin sequence appears consistent with formation of an α-helical conformation. For example and non-limiting, the Naja atra peptide is a helical cathelicidin.

A. Cathelicidins from a Variety of Host Organisms

Cathelicidin sequences have been isolated from a variety of host organisms, including but not limited to rabbit, canine, sheep, reptile, human, bovine, and porcine animals.

### 1. Rabbit Cathelicidin

CAP-18 encompasses a short stretch of amino acids isolated from rabbit granuloctyes. The N-terminal of the pro peptide is homlogous to what is observed in other members of the cathelicidin family of peptides (42). The C-terminal of the pro peptide, which is considered the mature peptide after proteolytic cleavage, is known to bind LPS. The LPS binding ability is relegated to the N-terminal residues of the mature CAP-18. In addition to the LPS binding ability the peptide is known to inhibit bacterial growth. CAP-18 proved effective against a variety of Gram-negative as well as Gram-positive bacterial species (43).

### 2. Canine Cathelicidin

Canines are considered highly resistant to infection by microorganisms. In 2007, the first naturally occurring carnivore cathelicidin was discovered in mature canine neutrophils (45). This 38-residue peptide is of similar size to a number of other peptides found in mammalian species and could be one of the reasons that carnivores have low incidences of diseases which are known to plague other mammalian species. The mature peptide has been shown to be the most potent antimicrobial peptide against N. gonorrhoea, the causative agent of a common human sexually transmitted disease (45). Researchers also reported that the peptide also had similar activity against Ureaplasma canigenitalium, a known sexually transmitted canine pathogen (45). This information coincides with the fact that though most canines are promiscuous, the level of STD's within these animals is almost non-existent. This cathelicidin contains a 29-residue signal peptide, a cathelin domain of 103 residues, and finally the previously mentioned 38-residue mature peptide (45). The structure of the mature peptide is similar to that seen is other cathelicidins and assumes an inducible α-helical conformation. This linear α-helical structure is critical for the peptide interaction with bacterial membranes. A final point which may play a role in the potency of this peptide is that the residues are not salt sensitive, allowing the peptide to function in a variety of differing microenvironments (45). Furthermore, the peptide was shown to bind to bacterial LPS, a characteristic that is similar to a number of other cathelicidins.

### 3. Bovine Cathelicidin BMAP-28

Discovered in the mid 1990's the bovine myeloid antimicrobial peptide (BMAP-28) was shown to be another member of the cathelicidin family (46). The peptide has been shown to possess a broad spectrum of antimicrobial activity in vitro against a variety of bacteria and fungi (46). The structure of the mature peptide is comprised of an amphipathic α-helical conformation in the N-terminal residues (1-18) followed by a stretch of primarily hydrophobic tail residues that comprise residues (19-28) at the C-terminal of the peptide. It appears that the hydrophobic C-terminal is responsible for a degree of cytotoxicity against a variety of cell lines such as activated human lymphocytes and tumor cells (46, 47). Removal of the C-terminal through the creation of a synthetic peptide comprosed only of the N-terminal amphapathic helical region displayed decreased cytotoxicity against both respring and nonrespiring cell lines (47).

### 4. Sheep Cathelicidin

SMAP-29 is an antimicrobial peptide found in sheep leukocytes. Over the last decade this peptide has been the subject of intense research due to the potent antimicrobial properties the peptide possesses. Specifically, the peptide possesses potent killing ability against antibiotic resistant strains of Pseudomonas aeruginosa (51, 52). The peptide possesses hemolytic activity for human erythrocytes, can permeabilize E. coli inner and outer membranes, and is also known to induce a massive potassium efflux in Gram-negative and Gram-positive bacterial species (53). Researchers have found that the peptide contains two regions that bind E. coli LPS (53). The sites are located at the two ends of the molecule, RGLRRLGR at the N-terminal and VLRIIRIA at the C-terminal. These two sites act in a cooperative fashion with one another to bind LPS. The peptide binding of LPS is believed to cause a displacement of divalent cations leading to a displacement of LPS molecules and their acyl chains. This displacement may lead to changes in the outer membrane causing it to expand providing a greater surface area for an interaction of the amphipathic regions of SMAP-29 with the bacterial membrane (53, 54). This increased interaction between the peptide and bacterial membrane leads to increased death and may account for the fact that the published MIC's for this cathelicidin are among the lowest seen (50, 54). Based on a variety of different studies it has been shown that SMAP-29 is not a host specific peptide and has antimicrobial activity against pathogens found in a number of different species (50).

### 5. Porcine Cathelicidin

Discovered in 1994, PMAP-37 is a component of the innate immune system of pigs (57). Cloning of cDNA from pig bone marrow was carried out and researchers found that a novel polypeptide with 167 residues contained a great deal of homology with cathelicidins previously discovered in a variety of other mammals. The peptide assumes the typical α-helical conformation observed in many linear cathelicidins. An interesting point regarding this peptide is the fact that a central stretch of 18 residues (15-32) contains a high degree of similarity with a similar stretch of residues (4-21) in cecropin B of Drosophila melanogaster (57).

The antimicrobial properties of PMAP-37 are attributed to the previously mentioned helical conformations interaction with bacterial membranes. With respect to all of the porcine cathelicidins, PMAP-23, PMAP-36, and PMAP-37, PMAP-37 has the highest hydrophobicity and the lowest positive charge. PMAP-37 contains an N-terminal helix followed by a C-terminal hydrophobic tail (58). Little structural information has been revealed regarding activity PMAP-37, with the majority focused on PMAP-23 and PMAP-36, but some researchers have suggested that the sequence and design of the peptide correspond with the typical α-helical amphipathic membrane killing mechanism that is commonly seen in other PMAP's and cathelicidins (57).

### 6. Human Cathelicidin

LL-37 is the only member of the cathelicidin family of antimicrobial peptides that is expressed in humans. The mature 37 residue peptide is produced via cleavage of the C-terminus of the hCAP-18 precursor protein. At physiological pH, the peptide contains a charge of +6 and is composed primarily of basic and hydrophobic residues. Upon contact with lipid membranes the peptide assumes an α-helical amphipathic conformation and this conformation and subsequent interaction if the basis for the antimicrobial effects commonly seen (59). Expression of LL-37 occurs in cell types such as neutrophils, monocytes, NK cells, T cells, and B cells as well as in the epithelial cells of the skin, testes, gastrointestional and respiratory tracts.

Research has shown that LL-37 possesses a wide range of biological functions other than antimicrobial activity. For example the peptide has been liked to prevention of P. aeruginosa biofilms, chemotaxis, mast cell degranulation, induction of immune functions, wound healing, apoptosis, angiogenesis, and finally regulation of the inflammatory response (59).

In addition to these abilities, LL-37 has also been shown to interact with cell membranes in such a way as to enter the cytosol of target cells through the possible alteration of membrane dynamics (60, 61). In murine models, LL-37 possesed the ability to bind and neutralize LPS, protecting the mice from endotoxic shock (62). The binding of LPS may be one of the ways in which the peptide enhances its antimicrobial ability by further promoting its interaction with the negatively charges bacterial membrane through its initial interaction/binding of the bacterial LPS. The physiological activity of the peptide is concentration dependant and determination of the actual in vivo concentrations has proven difficult for researchers. The level of the peptide in airway fluids is estimated to be approximately 2 μg/mL in adults and 5 μg/mL in neonates, with upregulation of those levels occurring during pulmonary infections (63-65). During cases of acute inflammation, such as psoriasis, the levels of LL-37 can reach as high at 1.5 mg/mL (66). Regardless, LL-37 is considered to antimicrobial in the phagolysosomes of immune cells, and at the sites of inflammation while at the same time playing a broader role in immunomodulation in systemic settings.

### 7. Reptile Cathelicidins

Recently cathelicidins have been discovered in various species of elapid snakes through the investigation into the cDNA libraries of the snakes venom glands (56). Naja atra, Bugarus fasciatus, and Ophiophagus hanna make up the three different species from which the first reptile cathelicidins were discovered (56). The mature peptide sequences differed only slightly among the different species and researchers tested the hemolytic and antimicrobial activity of the peptide produced by O. hanna or OH-CATH in their 2008 paper (56). OH-CATH showed no hemolytic activity against erythrocytes even at the highest concentrations of 200 μg/mL. In addition, the peptide proved to be an excellent inhibitor of bacterial growth and possessed broad spectrum activity even against bacterial isolates that were known to be multi-drug resistant. Research has shown that the full length snake peptide possess greater potency against a variety of known human pathogens, such as P. aeruginosa, than LL-37 does (56).

### B. Novel Reptile Cathelicidin Peptides

The present inventors discovered the antimicrobial activity of full-length Naja atra cathelicidin (NA-CATH) and four novel peptides based on the sequences of NA-CATH. In their paper, Zhao et al. noted that the three elapid snake cathelicidins shared a repeated 9-residue consensus sequence [6]. Here, the present inventors identified a broader repeated 11-residue sequence pattern (KR(F/A)KKFFKK(L/R)K), unique to Naja atra, denoted the ATRA motif A series of 11-residues peptides were designed in order to probe the significance of the conserved residues within the ATRA motif, and their contributions to the antimicrobial performance of NA-CATH.

The first peptide (ATRA-1: KRFKKFFKKLK), corresponds to the first 11 residues of NA-CATH, and the second peptide (ATRA-2: KRAKKFFKKPK) reflected residues 16-26 of the full-length peptide. They differ only by two residues: F/A at the third position and L/P at the tenth. The side-chain of alanine is much smaller than that of phenylalanine, which results in a loss in hydrophobic surface area. This may impact the ability of the ATRA-2 peptides to interact with the lipid bilayer of bacterial membranes. Proline tends to destabilize and disrupt helical structure, which may negatively impact the ability of ATRA-2 to attain a helical conformation when interacting with membranes. Either or both of the substitutions in ATRA-2 could reduce its potency relative to ATRA-1. To better understand how these substitutions affect peptide performance, two additional peptides (ATRA-1A and ATRA-1P) were designed based on ATRA-1 by replacing either the third residue with an alanine or the tenth residue with a proline respectively. All of the 11-residue peptides had C-terminal amide groups, which resulted in the peptides having a nominal charge of +8 under physiological conditions.

### C. Cathelicidin Sequences

The present disclosure contemplates cathelicidin sequences from a variety of host organisms, such as rabbit, canine, sheep, reptile, human, bovine, and porcine animals. Also contemplated are analogs, derivatives, variants, and functional fragments of the present cathelicidins, provided that the analogs, derivatives, variants, or functional fragments have detectable antimicrobial and or antibiofilm activity. It is not necessary that the analog, derivative, variant, or functional fragment have activity identical to the activity of the peptide from which the analog, derivative, variant, or functional fragment derives.

For a given sequence, one of ordinary skill in the art can readily identify conserved amino acids and non-conserved amino acids. Using a sequence analysis tool, such as BLAST, one of ordinary skill in the art can readily identify which amino acid(s) may be modified, removed, or substituted.

A cathelicidin functional fragment is a fragment of a larger cathelicidin sequence wherein the fragment confers antimicrobial, antibacterial, bactericidal, bacteriostatic and/or antibiofilm properties. For example, the term “variant” includes a cathelicidin functional fragment produced by the method disclosed herein in which at least one amino acid (e.g., from about 1 to 10 amino acids) of a reference peptide is substituted with another amino acid. The term “reference” peptide means any of the cathelicidin functional fragments of the disclosure

The disclosure also includes peptides that are variants of peptides exemplified herein. Examples of variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. Variant also encompasses a peptide having a substituted amino acid in place of an unsubstituted parent amino acid; typically, antibodies raised to the substituted peptide or polypeptide also specifically bind the unsubstituted peptide or polypeptide

Cathelicidin functional fragment can be identified by screening a large collection, or library, of random peptides or polypeptides using, for example, an animal model. Peptide libraries include, for example, tagged chemical libraries comprising peptides and peptidomimetic molecules. Peptide libraries also comprise those generated by phage display technology. Phage display technology includes the expression of peptide molecules on the surface of phage as well as other methodologies by which a protein ligand is or can be associated with the nucleic acid encoding it. These or other known methods can be used to produce a phage display library, from which the displayed peptides can be cleaved and assayed for, e.g., antibacterial activity. If desired, a population of peptides can be assayed for activity, and an active population can be subdivided and the assay repeated in order to isolate an active peptide from the population. Other methods for producing peptides useful in the disclosure include, for example, rational design and mutagenesis based on the amino acid sequences of a cathelicidin functional fragment.

A cathelicidin functional fragment can be a peptide mimetic, which is a non-amino acid chemical structure that mimics the structure of, for example, a cathelicidin functional fragment from a reptile, yet retains antimicrobial/antibacterial/antibiofilm properties. Such a mimetic generally is characterized as exhibiting similar physical characteristics such as size, charge or hydrophobicity in the same spatial arrangement found in the cathelicidin functional fragment counterpart. A specific example of a peptide mimetic is a compound in which the amide bond between one or more of the amino acids is replaced, for example, by a carbon-carbon bond or other bond well known in the art.

Peptides of the disclosure can be synthesized by commonly used methods such as those that include t-BOC or FMOC protection of alpha-amino groups. Both methods involve stepwise synthesis in which a single amino acid is added at each step starting from the C terminus of the peptide (See, Coligan, et al., Current Protocols in Immunology, Wiley Interscience, 1991, Unit 9). Peptides of the disclosure can also be synthesized by the well known solid phase peptide synthesis methods such as those described by Merrifield, J. Am. Chem. Soc., 85: 2149, 1962; and Stewart and Young, Solid Phase Peptides Synthesis, Freeman, San Francisco, 1969, pp. 27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0. degree. C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

The disclosure also includes isolated polynucleotides (e.g., DNA, cDNA, or RNA) encoding the peptides of the disclosure. Included are polynucleotides that encode analogs, mutants, and variants, of the peptides described herein. The term “isolated” as used herein refers to a polynucleotide that is substantially free of proteins, lipids, and other polynucleotides with which an in vivo-produced polynucleotide naturally associates. Typically, the polynucleotide is at least 70%, 80%, or 90% isolated from other matter, and conventional methods for synthesizing polynucleotides in vitro can be used in lieu of in vivo methods.

By taking into account the degeneracy of the genetic code, one of ordinary skill in the art can readily synthesize polynucleotides encoding the peptides of the disclosure. The polynucleotides of the disclosure can readily be used in conventional molecular biology methods to produce the peptides of the disclosure.

### D. Nucleic Acid Constructs

The present disclosure includes recombinant constructs comprising one or more of the nucleic acid or amino acid sequences disclosed herein. The constructs typically comprise a vector, such as a plasmid, a cosmid, a phage, a virus, a bacterial artificial chromosome (BAC), a yeast artificial chromosome (YAC), or the like, into which a nucleic acid sequence has been inserted, in a forward or reverse orientation. In a preferred embodiment, the construct further comprises regulatory sequences, including, for example, a promoter operably linked to the sequence. Large numbers of suitable vectors and promoters are known and are commercially available.

Recombinant nucleic acid constructs may be made using standard techniques. For example, a nucleotide sequence for transcription may be obtained by treating a vector containing said sequence with restriction enzymes to cut out the appropriate segment. The nucleotide sequence for transcription may also be generated by annealing and ligating synthetic oligonucleotides or by using synthetic oligonucleotides in a polymerase chain reaction (PCR) to give suitable restriction sites at each end. The nucleotide sequence then is cloned into a vector containing suitable regulatory elements, such as upstream promoter and downstream terminator sequences.

Typically, vectors include one or more cloned coding sequence (genomic or cDNA) under the transcriptional control of 5′ and 3′ regulatory sequences and a selectable marker. Such vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

The vector may also contain termination sequences, which are positioned downstream of the nucleic acid molecules of the invention, such that transcription of mRNA is terminated, and polyA sequences added. Exemplary terminators are the cauliflower mosaic virus (CaMV) 35S terminator and the nopaline synthase gene (NOS) terminator.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art.

### E. Antimicrobial Assay

The antimicrobial activity of a given peptides can be determined using a variety of methods known in the art. For example, antimicrobial activity can be determined using conventional methods, such as “minimal inhibitory concentration (MIC),” whereby the lowest concentration at which no change in OD is observed for a given period of time is recorded as the MIC. Alternatively, a “fractional inhibitory concentration (FIC)” assay can be used to measure synergy between the peptides of the disclosure, or the peptides in combination with known antibiotics. FICs can be performed by checkerboard titrations of peptides in one dimension of a microtiter plate, and of antibiotics in the other dimension, for example.

For a given peptide, antimicrobial activity can be determined as a function of bacterial survival based on the ratio of the number of colonies on the plates corresponding to the peptide concentration and the average number of colonies observed for assay cultures lacking peptide. The peptide concentration required to kill 50% of the viable bacteria in the assay cultures (EC50) can be determined by plotting percent mortality as a function of the log of peptide concentration (log μg/ml) and fitting the data using methods readily known in the art.

### F. Antibiofilm Assay

The antibiofilm activity of a given peptides can be determined using a variety of methods known in the art. For example, antibiofilm activity can be determined using conventional methods, such as the inhibition of the formation of biofilm as measured by crystal violet staining. Alternatively, an inhibition of biofilm in a flow cell or a glass chambered slide can be performed and measured using Confocal microscopy. The corresponding effect of the peptide on bacterial growth (separate from biofilm formation) can be determined by measuring the “minimal inhibitory concentration (MIC), whereby the lowest concentration at which no change in OD is observed for a given period of time is recorded as the MIC. Alternatively, a “fractional inhibitory concentration (FIC)” assay can be used to measure synergy between the peptides of the disclosure, or the peptides in combination with known antibiotics. FICs can be performed by checkerboard titrations of peptides in one dimension of a microtiter plate, and of antibiotics in the other dimension, for example.

For a given peptide, antibiofilm activity can be determined as a function of the amount of crystal violet staining in a treatment well of a 96 well plate compared to untreated wells and control wells. The peptide concentration required to inhibit 50% of the biofilm formation can be determined by calculation of percent inhibition compared to the log of the peptide concentration. The peptide concentration required to kill 50% of the viable bacteria in the biofilm (EC50) can be determined by plotting percent mortality as a function of the log of peptide concentration (log μg/ml) and fitting the data using methods readily known in the art.

### G. Illustrative Products

The disclosure also provides methodology and products for inhibiting microbial infection using an inhibiting effective amount of a cathelicidin. For example, by adding a cathelicidin, or functional fragment thereof, to a culture comprising a microorganism, one can measure the susceptibility of a culture to said microorganism. Alternatively, inhibiting can occur in vivo, for example, by administering a cathelicidin, or a functional fragment thereof, to a subject susceptible to or afflicted with a microbial infection. A cathelicidin functional fragment(s) of the disclosure can be administered to any host, including a human or non-human animal, in an amount effective to inhibit growth of a microorganism.

An illustrative cathelicidin, or a functional fragment thereof, may be useful as a broad-spectrum antimicrobials suitable for tackling the growing problem of antibiotic-resistant bacteria strains, and for treating and/or preventing outbreaks of infectious diseases, including diseases caused by bioterrorism agents like anthrax, plague, cholera, gastroenteritis, multidrug-resistant tuberculosis (MDR TB), as well as oral diseases, as periodontal diseases.

Likewise, a cathelicidin, or functional fragment thereof, can be used be used therapeutically and/or prophylactically as a means for defense against a biological warfare. For example, the disclosure contemplates kits comprising formulations comprising a cathelicidin, or functional fragment thereof. Such a formulation could be applied/administered either before (prophylactic) during, or after exposure to a microorganism, thereby inducing a subject's natural cathelicidin activity.

Any of a variety of art-known methods can be used to administer a cathelicidin, or functional fragment thereof, to a subject. For example, a composition can be administered parenterally by injection or by gradual infusion over time. Likewise, a cathelicidin, or functional fragment thereof, can be administered intravenously, intraperitoneally, intramuscularly, subcutaneously, intracavity, or transdermally. Such cathelicidin may be formulated for topical administration (e.g., as a lotion, cream, spray, gel, or ointment). Examples of formulations in the market place include topical lotions, creams, soaps, wipes, and the like. It may be formulated into liposomes to reduce toxicity or increase bioavailability. Other delivery methods include oral methods that entail encapsulation of the peptide in microspheres or proteinoids, aerosol delivery (e.g., to the lungs), or transdermal delivery (e.g., by iontophoresis or transdermal electroporation). Methods of administration are known and readily available those ordinarily skilled in the art. The cathelicidin, or functional fragment thereof, can be used, for example, for sterilizing materials susceptible to microbial contamination. For example, the peptides can be used as preservatives in processed foods, or as spray disinfectants commonly used in the household or clinical environment. The optimal amount of a cathelicidin peptide of the disclosure for any given application can be readily determined by one of ordinary skill in the art.

The following examples are included to demonstrate preferred embodiments. However, those of ordinary skill in the art should, in light of the present disclosure, appreciate that many changes can be made in the specific embodiments which are disclosed and still obtain a like or similar result without departing from the spirit and scope of the disclosure.

### Example 1

### Antimicrobial Activity of Cathelicidins from Various Organisms

A. Bacterial Strains and Media for A. actinomycetemcomitans and E. coli Assays

A. actinomycetemcomitans Y4 (serotype b) was used in this study and was grown in Todd-Hewitt broth (Difco Laboratories, Detroit, Mich.) at 37° C. for 24 hours in an atmosphere of 5% CO2 in air. A. actinomycetemcomitans was plated on Brain Heart Infusion Agar (Difco Laboratories, Detroit, Mich.).

K12 E. coli (ATCC #25404) was purchased from the American Type Culture Collection (Manassas, Va., USA). The bacterial strain was grown to mid-logarithmic phase in Luria Bertani broth (Difco Laboratories, Detroit, Mich.) at 37° C. for 24 hours. E. coli was plated on Luria Bertani Agar plates.

### B. Circular Dichroism:

Circular dichroism (CD) spectra of the full-length N. atra cathelicidin and the truncated peptides were collected using a Jasco J-815 spectropolarimeter. Samples were allowed to equilibrate for 10 minutes at 25° C. prior to data collection, and the temperature in the chamber was maintained at a constant 25° C. throughout each scan. Spectra were collected from 190 to 240 nm using 0.1 nm intervals, and a total of 4 scans per sample were performed and averaged using a cuvette with a path-length of 0.1 cm. All peptides were analyzed at a concentration of 200 μg/mL in either 10 mM sodium phosphate (pH 7) or 90 mM sodium dodecyl sulfate (SDS).

### C. Antimicrobial Assay:

Antimicrobial assays were performed following previously published protocols with modification [8, 9]. The microorganisms were grown to mid-logarithmic phase in appropriate broth and then diluted to 10E6 CFU/ml in 10 mM potassium phosphate-1% trypticase soy broth (for E. coli) or 5% Todd-Hewitt broth (for A. actinomycetemcomitans) and a pH 7.4. Alternatively, the bacteria were grown to mid-log phase, prepared as frozen stocks with 20% glycerol and the frozen stocks enumerated by CFU plating so that a known number of bacteria are added to each well of the experiment.

Bacteria (50 μl) were incubated in the presence of different concentrations of peptide, from 10E-7 to 10E3 μg/ml. Assays were incubated at 37° C. for 3 h in 5% CO2, after which serial dilutions were prepared in 1× Dulbecco's Phosphate buffered Saline and then plated in triplicate onto appropriate agar plates. The plates were incubated at 37° C. overnight, and the colonies were counted after 16 hrs for E. coli and 24 hours for A. actinomycetemcomitans.

Bacterial survival at each peptide concentration was calculated based on the ratio of the number of colonies on the plates corresponding to the peptide concentration and the average number of colonies observed for assay cultures lacking peptide. The peptide concentration required to kill 50% of the viable bacteria in the assay cultures (EC50) was determined by plotting percent mortality as a function of the log of peptide concentration (log μg/ml) and fitting the data, using GraphPad Prism (GraphPad Software Inc., San Diego, Calif., USA), to Equation (1), which describes sigmoidal dose-response:

Y=Bottom+((Top-Bottom)/(1+10̂[(Log EC50−X)*Hill Slope] Equation 1

Where Y corresponds to bacterial mortality (%) at a given peptide concentration (ug/ml), with X being the logarithm of that concentration (log μg/ml). In the equation, “Top” and “Bottom” refer to the upper and lower boundaries, and were constrained to values <100% and >0%, respectively. For the purpose of graphing, samples that had no peptide are plotted at 10̂-9 μg/ml. EC50 values were determined by fitting the data from the antimicrobial assays to a standard sigmoidal dose-response curve (Eq. (1)). Errors were reported based on the standard deviation from the mean of the Log EC50 values.

### D. Hemolysis Assay:

Hemolytic activities of the peptides were determined using horse erythrocytes (Hemasource Inc. Eugene, Oreg., USA) in an assay adapted to a microtiter plate format [8]. Erythrocytes were prepared by centrifuging 1 ml of horse blood at 1620 g for 10 min, and then re-suspending the pelleted cells in 1 ml of 1× dPBS. The cells were then pelleted again and the process was repeated three more times. Following the final wash the cells were re-suspended in 750 ml of dPBS. Two hundred microliters of washed erythrocyte suspension was then diluted in 9800 μl of dPBS to afford a 2% suspension.

Aliquots of 50 μl of sterile water, peptide and dPBS were then combined in the wells of a u-shaped 96-well microtiter plate so as to provide a gradient of peptide concentration (0, 0.1, 1, 10, 100, and 1000 μg/ml), to which 50 μl of 2% erythrocyte was added. The assay solutions were then incubated at 37° C. with 5% CO2 for 1 h. An additional 100 μl of phosphate buffer was then added to each well, and the microtiter plate was centrifuged at 1000×g for 2 min to pellet cells and debris. An aliquot of supernatant (150 μl) from each well was then transferred to a fresh flat bottom microtiter plate, and the absorbance at 540 nm (heme) was obtained for each solution. The percent hemolysis was calculated based on the ratio of the absorption of supernatants from wells containing peptide and the absorption of supernatants from wells containing no peptide.

### E. Statistical Analysis:

Antimicrobial assay measurements were performed in triplicates. Standard deviations of the mean of each set are represented on each graph. Where the error bars can not be seen, the error is very small.

### Example 2

### Rabbit Cathelicidin CAP-18

CAP-18 encompasses a short stretch of amino acids isolated from rabbit granuloctyes. Following the methodology disclosed above in Example 1, A. actinomycetemcomitans was incubated with varying concentrations of CAP-18.

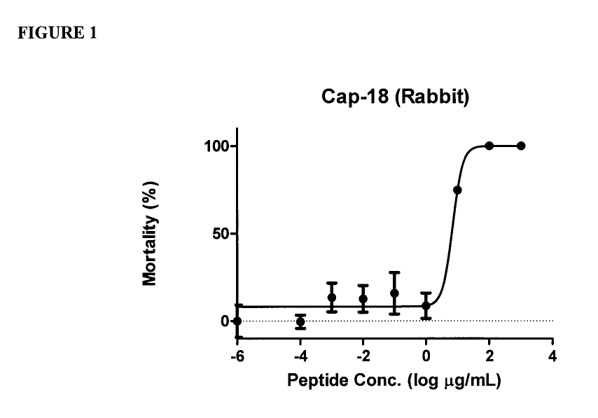

As shown in FIG. 1, The EC50 value was determined to be 7.049 μg/mL, the highest value observed among the full length cathelicidin peptides. The potency, based on the EC50, of this peptide was considerably lower than what was seen in other peptides allowing for the conclusion that this peptide would be excluded from further testing based on the results obtained.

### Example 3

### Canine Cathelicidin K9CATH

Following the methodology disclosed above in Example 1, the susceptibility of A. actinomycetemcomitans to various concentrations of K9CATH peptide was tested.

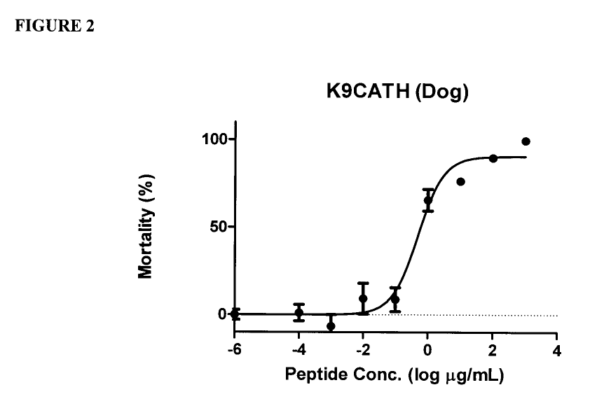

As shown in FIG. 2, the EC50 values was determined by statistical analysis to be 0.5005 μg/mL. Of all the peptides and antibiotics screened K9CATH proved to be one of the most antimicrobial.

### Example 4

### Bovine Cathelicidin BMAP-28

Following the methodology disclosed above in Example 1, the susceptibility of A. actinomycetemcomitans to various concentrations of BMAP-28 peptide was tested.

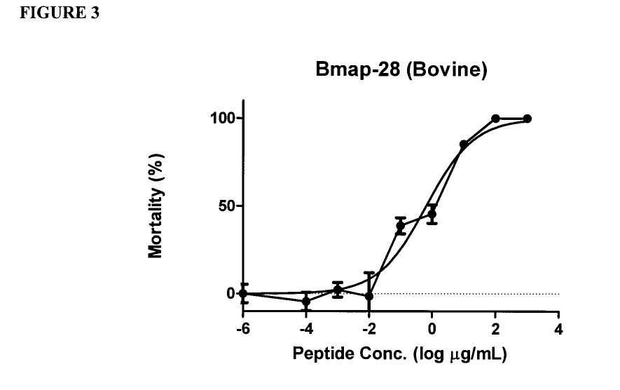

The lack of host specificity and the previous antimicrobial activity proved correct with the peptide inhibiting bacteria growth during an in vitro study. As shown in FIG. 3, statistical analysis of the experimental data produced an EC50 value of 0.6616 μg/mL, making it one of the most potent peptides tested.

### Example 5

### Sheep Cathelicidin SMAP-29

Following the methodology disclosed above in Example 1, the susceptibility of A. actinomycetemcomitans to various concentrations of SMAP-29 peptide was tested.

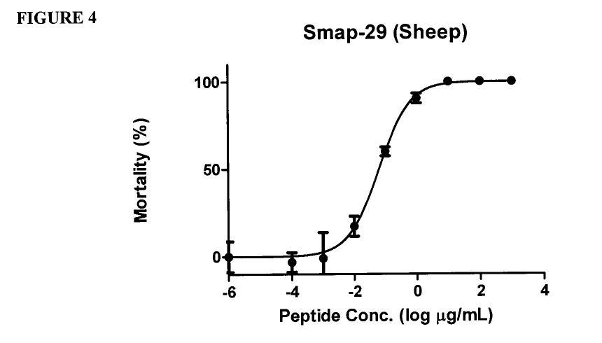

The data produced in this study correlates with the initial study and at a concentration of 10 μg/mL there was 99% inhibition of the bacteria, while at a concentration of 1 μg/mL the inhibition of the bacteria was approximately 90%. As shown in FIG. 4, the calculated EC50 for SMAP-29 was 0.06386 μg/mL, the lowest of any of the antimicrobial agents tested.

### Example 6

### Porcine Cathelicidin PMAP-37

Following the methodology disclosed above in Example 1, the susceptibility of A. actinomycetemcomitans to various concentrations of PMAP-37 peptide was tested.

PMAP-37 peptide possessed strong activity against a number of previously mentioned bacterial strains, for example, MIC values for E. coli (ATCC 25922) and P. aeruginosa (ATCC 27853) were 1 μM and 4 μM. However more recently scientists have focused their time and effort on better understanding the shorter porcine cathelicidins, PMAP-23 and PMAP-36, so little structural or antimicrobial data has been presented regarding the activity of PMAP-37.

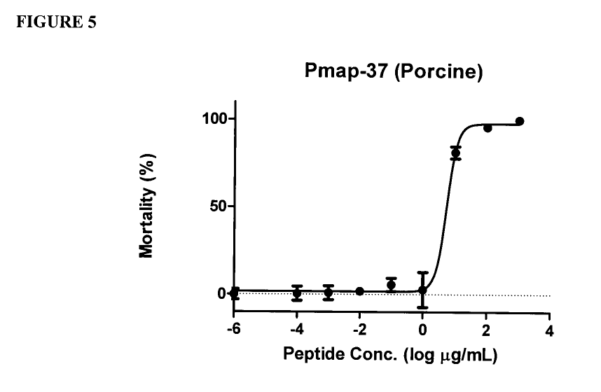

As shown in FIG. 5, PMAP-37 is a decent inhibitor of A. actinomycetemcomitans Y4. The EC50 value was determined to be 5.465 μg/mL. This value was approximately a hundred fold higher than the smallest EC50.

### Example 7

### Human Cathelicidin LL-37 and LL-37 Pentamide

This current study addresses the susceptibility of A. actiniomycetemcomitans to both conventional LL-37 as well as a peptide similar to LL-37 termed LL-37 Pentamide. Differences between the two peptides result from the fact that synthetic LL-37 pentamide has all of the negatively charged aspartic acid residues replaced with neutrally charged asparagines and the negative glutamic acids are replaced with glutamines. The removal of the acidic residues produces a synthetic peptide with a greater positive charge which hypothetically should increase the ability of the peptide to interact with negatively charged bacterial membranes. LL-37 pentamide retains its overall structure with the substitutions and has been shown to have increased antibacterial properties when compared to LL-37 in vitro against strains of Staphyolcoccus (67).

A variety of research groups have previously attempted to quantify the potency of LL-37 and LL-37 pentamide against A. actinomycetemcomitans, but their results all differed from one another in such a way that it seemed necessary to further investigate the antimicrobial activity of these peptides. Current published information has stated that the ED99 of LL-37 in vitro against A. actinomycetemcomitans is 8.2 μg/mL (68), while another published the MIC as 100 mg/L (69), and finally another research team reported that the MIC was >200 μg/mL in their published paper (70). Only one experiment has been run using LL-37 pentamide and the published ED99 was 8.7 μg/mL (68).

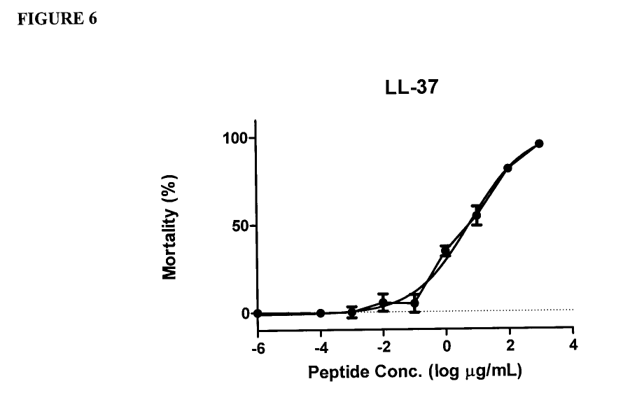

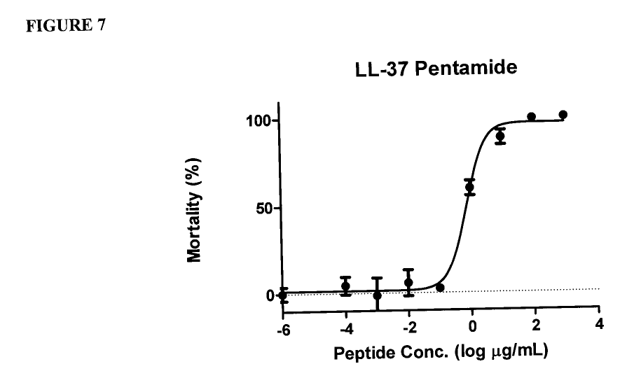

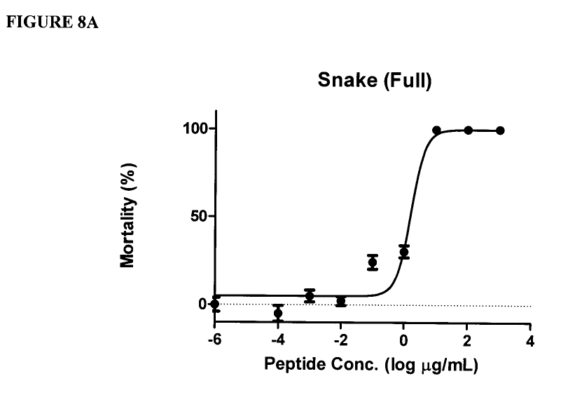

Results gathered in this study revealed an EC50 of 6.224 μg/mL for LL-37. All of the previous experiments were run using various procedures, but the closest protocols determined the MBC to be above 100 μg/mL, a number that coincides with what can be seen in FIG. 6. Only at the highest concentration was the peptide able to inhibit A. actinomycetemcomitans Y4. At all other concentrations up to 0.1 μg/mL, LL-37 was able to kill a portion of the bacteria, but not completely inhibit it like what was seen in some other peptides. A. actinomycetemcomitans proved to be more susceptible to LL-37 Pentamide, as shown in FIG. 7, with a EC50 value of 0.7648 μg/mL. T his difference in EC50 values may be as a result of the differences in charge between the synthetic LL-37 Pentamide and LL-37. The replacement of the negative residues with neutral ones boosts the charge of LL-37 Pentamide to +11, a fact that may increase its ability to interact with the bacterial membrane and induce death. LL-37 pentamide was one of the more potent antimicrobial agents tested in this study.

### Example 8

### Reptile Cathelicidins

The present inventors discovered the antimicrobial activity of full-length Naja atra cathelicidin (NA-CATH) and four novel peptides based on the sequences of NA-CATH. In their paper, Zhao et al. noted that the three elapid snake cathelicidins shared a repeated 9-residue consensus sequence [6]. Here, the present inventors identified a broader repeated 11-residue sequence pattern (KR(F/A)KKFFKK(L/R)K), unique to Naja atra, denoted the ATRA motif A series of 11-residues peptides were designed in order to probe the significance of the conserved residues within the ATRA motif, and their contributions to the antimicrobial performance of NA-CATH.

The first peptide (ATRA-1: KRFKKFFKKLK), correspond to the first 11 residues of NA-CATH, and the second peptide (ATRA-2: KRAKKFFKKPK) reflected residues 16-26 of the full-length peptide. They differ only by two residues: F/A at the third position and L/P at the tenth. The side-chain of alanine is much smaller than that of phenylalanine, which results in a loss in hydrophobic surface area. This may impact the ability of the ATRA-2 peptides to interact with the lipid bilayer of bacterial membranes. Proline tends to destabilize and disrupt helical structure, which may negatively impact the ability of ATRA-2 to attain a helical conformation when interacting with membranes. Either or both of the substitutions in ATRA-2 could reduce its potency relative to ATRA-1. To better understand how these substitutions affect peptide performance, two additional peptides (ATRA-1A and ATRA-1P) were designed based on ATRA-1 by replacing either the third residue with an alanine or the tenth residue with a proline respectively. All of the 11-residue peptides had C-terminal amide groups, which resulted in the peptides having a nominal charge of +8 under physiological conditions.

The antimicrobial activity of full-length NA-CATH and the novel 11-residue ATRA motif peptides were assessed against E. coli and A. actinomycetemcomitans, both gram-negative organisms. Many CAMPs are effective at killing bacteria but also lyse host cells. Therefore, hemolytic assays were used to assess the propensity of the peptides to lyse mammalian cells. Finally, the helicities of full-length Naja atra cathelicidin and the novel ATRA motif peptides were evaluated using circular dichroism in conditions simulating bacterial membranes.

### Example 9

### Antimicrobial Activity of Full-Length Naja atra Cathelicidin (NA-CATH) and Four Novel Peptides Based on the Sequences of NA-CATH

A. Bacterial Strains and Media for A. actinomycetemcomitans and E. coli Assays

A. actinomycetemcomitans Y4 (serotype b) was used in this study and was grown in Todd-Hewitt broth (Difco Laboratories, Detroit, Mich.) at 37° C. for 24 hours in an atmosphere of 5% CO2 in air. A. actinomycetemcomitans was plated on Brain Heart Infusion Agar (Difco Laboratories, Detroit, Mich.).

K12 E. coli (ATCC #25404) was purchased from the American Type Culture Collection (Manassas, Va., USA). The bacterial strain was grown to mid-logarithmic phase in Luria Bertani broth (Difco Laboratories, Detroit, Mich.) at 37° C. for 24 hours. E. coli was plated on Luria Bertani Agar plates.

### B. Circular Dichroism:

Circular dichroism (CD) spectra of the full-length N. atra cathelicidin and the truncated peptides were collected using a Jasco J-815 spectropolarimeter. Samples were allowed to equilibrate for 10 minutes at 25° C. prior to data collection, and the temperature in the chamber was maintained at a constant 25° C. throughout each scan. Spectra were collected from 190 to 240 nm using 0.1 nm intervals, and a total of 4 scans per sample were performed and averaged using a cuvette with a path-length of 0.1 cm. All peptides were analyzed at a concentration of 200 μg/mL in either 10 mM sodium phosphate (pH 7) or 90 mM sodium dodecyl sulfate (SDS).

### C. Antimicrobial Assay:

Antimicrobial assays were performed following previously published protocols with modification [8, 9]. Full-length NA-CATH and the four novel peptides were synthesized to order by Genscript USA, Inc. The microorganisms were grown to mid-logarithmic phase in appropriate broth and then diluted to 10E6 CFU/ml in 10 mM potassium phosphate-1% trypticase soy broth (for E. coli) or 5% Todd-Hewitt broth (for A. actinomycetemcomitans) and a pH 7.4. Alternatively, the bacteria were grown to mid-log phase, prepared as frozen stocks with 20% glycerol and the frozen stocks enumerated by CFU plating so that a known number of bacteria are added to each well of the experiment.

Bacteria (50 μl) were incubated in the presence of different concentrations of peptide, from 10E-7 to 10E3 μg/ml. Assays were incubated at 37° C. for 3 h in 5% CO2, after which serial dilutions were prepared in 1× Dulbecco's Phosphate buffered Saline and then plated in triplicate onto appropriate agar plates. The plates were incubated at 37° C. overnight, and the colonies were counted after 16 hrs for E. coli and 24 hours for A. actinomycetemcomitans.

Bacterial survival at each peptide concentration was calculated based on the ratio of the number of colonies on the plates corresponding to the peptide concentration and the average number of colonies observed for assay cultures lacking peptide. The peptide concentration required to kill 50% of the viable bacteria in the assay cultures (EC50) was determined by plotting percent mortality as a function of the log of peptide concentration (log μg/ml) and fitting the data, using GraphPad Prism (GraphPad Software Inc., San Diego, Calif., USA), to Equation (1), which describes sigmoidal dose-response:

Y=Bottom+((Top-Bottom)/(1+10̂[(Log EC50−X)*Hill Slope] Equation 1

Where Y corresponds to bacterial mortality (%) at a given peptide concentration (ug/ml), with X being the logarithm of that concentration (log μg/ml). In the equation, “Top” and “Bottom” refer to the upper and lower boundaries, and were constrained to values <100% and >0%, respectively. For the purpose of graphing, samples that had no peptide are plotted at 10̂-9 μg/ml. EC50 values were determined by fitting the data from the antimicrobial assays to a standard sigmoidal dose-response curve (Eq. (1)). Errors were reported based on the standard deviation from the mean of the Log EC50 values.

### D. Hemolysis Assay:

Hemolytic activities of the peptides were determined using horse erythrocytes (Hemasource Inc. Eugene, Oreg., USA) in an assay adapted to a microtiter plate format [8]. Erythrocytes were prepared by centrifuging 1 ml of horse blood at 1620 g for 10 min, and then re-suspending the pelleted cells in 1 ml of 1× dPBS. The cells were then pelleted again and the process was repeated three more times. Following the final wash the cells were re-suspended in 750 ml of dPBS. Two hundred microliters of washed erythrocyte suspension was then diluted in 9800 μl of dPBS to afford a 2% suspension.

Aliquots of 50 μl of sterile water, peptide and dPBS were then combined in the wells of a u-shaped 96-well microtiter plate so as to provide a gradient of peptide concentration (0, 0.1, 1, 10, 100, and 1000 μg/ml), to which 50 μl of 2% erythrocyte was added. The assay solutions were then incubated at 37° C. with 5% CO2 for 1 h. An additional 100 μl of phosphate buffer was then added to each well, and the microtiter plate was centrifuged at 1000×g for 2 min to pellet cells and debris. An aliquot of supernatant (150 μl) from each well was then transferred to a fresh flat bottom microtiter plate, and the absorbance at 540 nm (heme) was obtained for each solution. The percent hemolysis was calculated based on the ratio of the absorption of supernatants from wells containing peptide and the absorption of supernatants from wells containing no peptide.

### E. Statistical Analysis:

Antimicrobial assay measurements were performed in triplicates. Standard deviations of the mean of each set are represented on each graph. Where the error bars can not be seen, the error is very small.

A series of helical AMPs shown in Table 1 below, including the human cathelicidin LL-37, the snake cathelicidin NA-CATH, and small peptides designed based on the repeated ATRA motif were assessed for their effectiveness against the gram-negative microbes E. coli and A. actinomycetemcomitans.

TABLE 1
Sequences of Antimicrobial Peptides

| | | Net |
|---|---|---|
| Peptide | Sequence | Charge |
| NA-CATH | KRFKKFFKKLKNSVKKRAKKFFKKPKVIGVTFPF | +15 |
| (Full) * | (ATRA-1) (ATRA-2) | |
| ATRA-1 | KRFKKFFKKLK-NH2 | +8 |
| ATRA-2 | KRAKKFFKKPK-NH2 | +8 |
| ATRA-1A | KRAKKFFKKLK-NH2 | +8 |
| ATRA-1P | KRFKKFFKKPK-NH2 | +8 |
| LL-37 | LLGDFFRKSKEKIGKEFKRIVQRIKDFLRNLVPR | +6 |
| | TES | |

* NA-CATH, [6].

The experimental techniques used herein were markedly different from methods previously reported for evaluating cathelicidin activities [10, 11] in which the MIC was determined measuring the optical density of cultures incubated with peptide, or agar diffusion assays. Here, bactericidal EC50 values were determined based on the enumeration of viable CFUs following co-incubation of peptide and bacteria in a phosphate-based solution. The scientific community recognizes both as acceptable measures of antimicrobial activity, but the enumeration of viable CFUs appears to provide a more accurate measure of microbicidal effectiveness. The results are presented in detail below, and are compared to results for LL37, a helical cathelicidin of similar length.

### Example 10

### Antimicrobial Activity of Full-Length Naja atra Cathelicidin, NA-CATH

Using the methodology described above in Example 1, the antimicrobial activity of the full-length Naja atm cathelicidin, NA-CATH, was determined for A. actinomycetemcomitans (Table 2) and E. coli (Table 3).

TABLE 2
EC50's of Antimicrobial Peptides against
A. actinomycetemcomitans Y4.

| Antimicrobial | Molecular | EC50 | | EC50 |
|---|---|---|---|---|
| Peptide | Weight | ug/mL | 95% CI | uM |
| LL-37 | 4493.3 | 6.24 | | 1.39 |
| NA-CATH Full | 4175.22 | 1.65 | | 0.396 |
| ATRA-1 | 1496.94 | 0.929 | | 0.621 |
| ATRA-2 | 1404.8 | 158 | | 113 |
| ATRA-1A | 1420.84 | 1.07 | | 0.755 |
| ATRA-1P | 1480.89 | 102 | | 68.8 |

TABLE 3
EC50's of Antimicrobial Peptides against E. coli.

| Antimicrobial | Molecular | EC50 | | EC50 |
|---|---|---|---|---|
| Peptide | Weight | ug/mL | 95% CI | uM |
| LL-37 | 4493.30 | 0.514 | | 0.114 |
| NA-CATH Full | 4175.22 | 0.192 | 0.0613-0.132 | 0.046 |
| ATRA-1 | 1496.94 | 0.881 | 0.539-1.44 | 0.589 |
| ATRA-2 | 1404.8 | 22.2 | 14.2-34.6 | 15.8 |
| ATRA-1A | 1420.84 | 0.932 | 0.726-1.20 | 0.659 |
| ATRA-1P | 1480.89 | 7.05 | 4.57-10.9 | 4.76 |

The NA-CATH peptide possesses a nominal charge of +15 at physiological pH. Published data indicated that the related elapid cathelicidin OH-CATH displayed potent antimicrobial activity against numerous microbes. MIC values for OH-CATH of 8 μg/mL against E. coli (ATCC 25922) and 2 μg/mL for P. aeruginosa PA01 were reported [6].

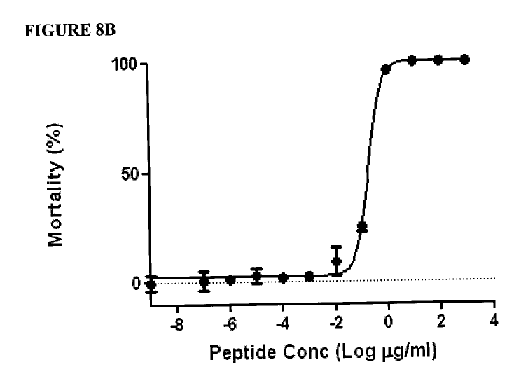

In the present disclosure, the conditions used to assess hCAMP activity measure microbicidal effectiveness, not inhibition of growth. For the full-length NA-CATH peptide, the EC50 value against the oral pathogen A. actinomycetemcomitans was found to be 1.65 μg/mL (FIG. 8A), and the EC50 value against E. coli K-12 strain was determined to be 0.1921 μg/mL (FIG. 8B). Using the same experimental conditions as used for NA-CATH, the effectiveness of LL-37 against E. coli K12 was found to be 0.519 μg/mL.

### Example 11

### Novel 11-Residue Peptides ATRA-1 and ATRA-2

Two novel 11-residue peptides ATRA-1 (KRFKKFFKKLK-NH₂) and ATRA-2 (KRAKKFFKKPK-NH₂) were designed based on the repeated ATRA motif from NA-CATH (Table 2).

These peptides are unique to the elapid snakes, although BLAST analysis of NCBI genomic databases revealed a similar sequence in the bovine cathelicidin 6/BMAP-27 (Differences in brackets: K(K)FKK(L)FKKL). The C-termini of ATRA-1 and -2 were amidated so as to eliminate the only acidic group in the sequence and maximize their overall positive charge. This resulted in both peptides having a nominal net charge of +8 at physiological pH.

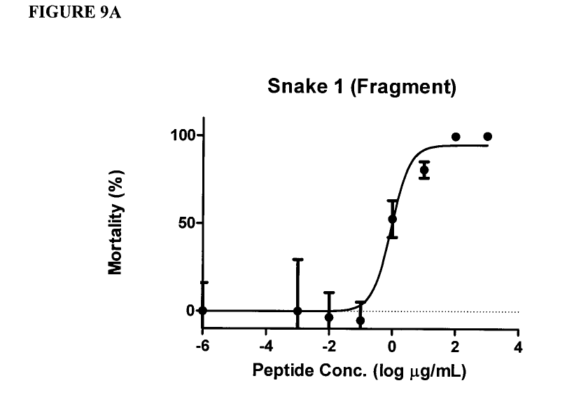

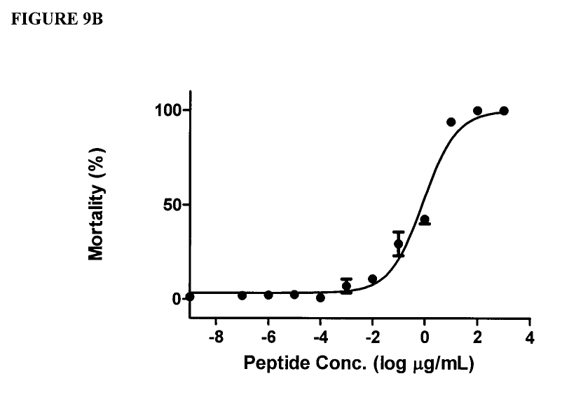

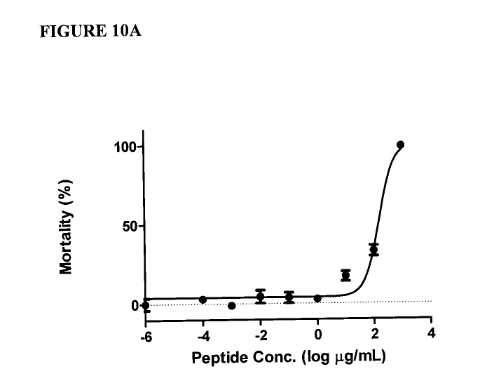

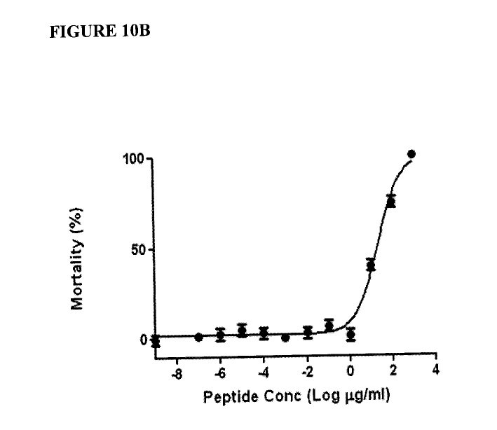

When assessed against A. actinomycetemcomitans, ATRA-1 and ATRA-2 were determined to have EC50 values of 0.926 and 158 μg/mL respectively. See FIG. 9A and FIG. 10A.

Against E. coli, ATRA-1 had an EC50 value of 0.881 μg/mL and ATRA-2 an EC50 of 22.2 μg/mL (FIG. 9B and FIG. 10B).

These 11-residue ATRA peptides have very similar sequences, differing only at positions 3 and 10, and similar overall positive charge, yet their potencies against A. actinomycetemcomitans differ by 200-fold on a molar basis, and against E. coli a 300-fold disparity (Table 1).

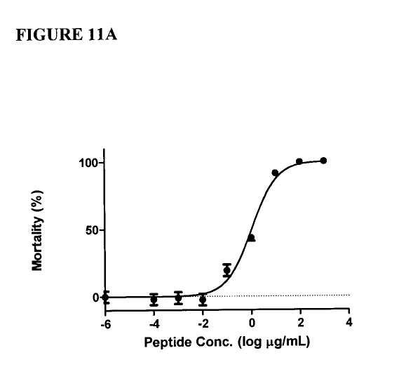

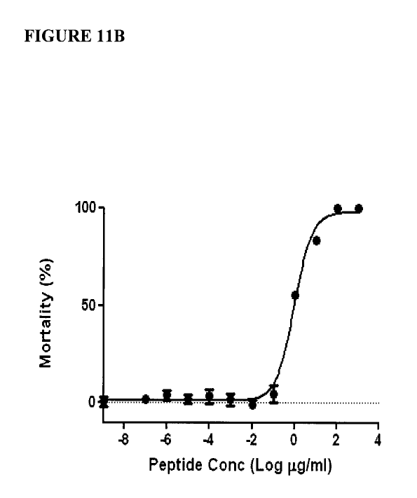

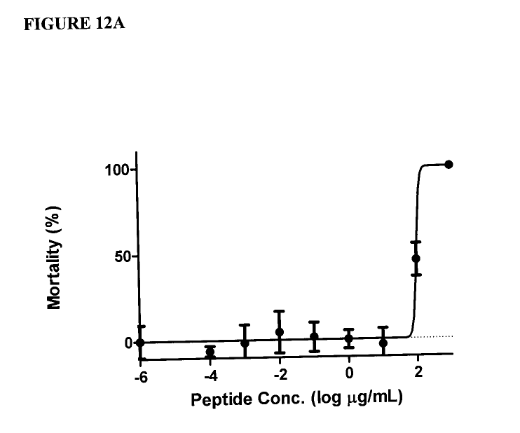

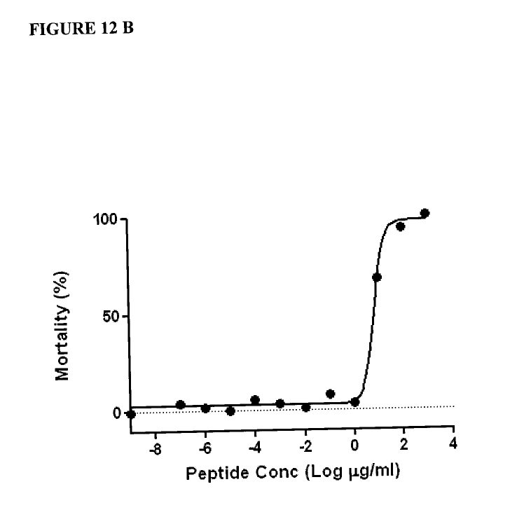

In order to explore how the differences between ATRA-1 and -2 at positions 3 and 10 contribute to the significant differences in their respective antimicrobial activities, a pair of intermediate peptides were designed based on the sequence differences between the peptides. In one peptide, ATRA-1A, the phenylalanine residue at position 3 of ATRA-1 was replaced with an alanine, and in the second peptide, ATRA-1P the leucine residue at position 10 of ATRA-1 was replaced with a proline residue. The antimicrobial activities of these peptides were assessed against both A. actinomycetemcomitans and E. coli. The EC50 values for ATRA-1A and ATRA-1P against A. actinomycetemcomitans were determined to be 1.07 and 102 μg/mL respectively (FIG. 11A and FIG. 12A). Similarly, the EC50 values of these peptides against E. coli were found to be 0.932 μg/mL for ATRA-1A and 7.05 μg/mL for ATRA-1P (FIG. 11B and FIG. 12B). Thus, these peptides display antimicrobial activities against E. coli that are between those of ATRA-1 and ATRA-2.

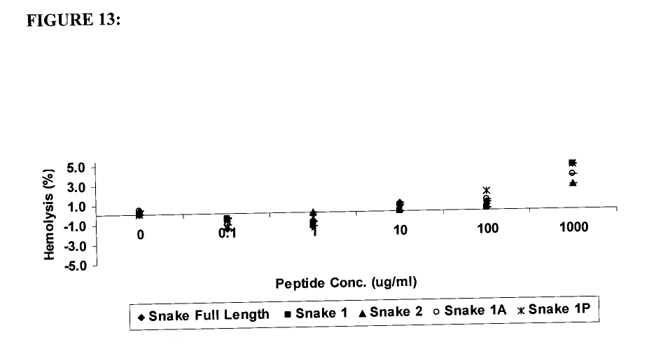

The hemolytic activity of each of the peptides was determined using 2% horse erythrocytes. As shown in FIG. 13, no hemolysis was evidenced by any of the peptides up to a peptide concentration of 10 ug/ml. At 100 ug/ml, ATRA-1A and ATRA-1P elicited 1.1% and 1.9% hemolysis respectively. At this concentration, full-length NA-CATH and ATRA-1 showed 0.9% hemolysis, and ATRA-2 had the lowest hemolysis (0.2%). At the very high, non-physiological concentration of 1000 ug/ml, up to 4.5% hemolysis was observed for full-length NA-CATH peptide, ATRA-1, and ATRA-1P, with ATRA-2 and ATRA-1A exhibiting 2.6% and 3.5% hemolysis respectively. Other studies only examine hemolytic activity up to 200 μg/ml [6].

### Example 12

### Predicted Helical Cathelicidins

Based on its amino acid sequence, the Naja atra peptide has been grouped with the helical cathelicidins, meaning that a large portion of its sequence appears consistent with formation of an α-helical conformation. Analysis of the amino acid sequences of LL-37, NA-CATH, and the truncated peptides using SSPro in the SCRATCH suite of protein analysis utilities [12], suggests that LL-37, full-length NA-CATH, ATRA-1 and ATRA-1A have significant propensities to attain α-helical structures, while ATRA-2 and ATRA-1P, are predicted to have significantly less helical structure. The results of these analyses are summarized in Table 4 below

TABLE 4
Predicted Helicity of Peptides.

| | | Nominal |
|---|---|---|
| | | Charge |
| Peptide | Sequence* | (pH7) |
| LL-37 | LLGDFFRKSKEKIGKEFKRIVQRIKDFLRNLVPR | +6 |
| | TES | |
| NA-CATH | KRFKKFFKKLKNSVKKRAKKFFKKPKVIGVTFPF | +15 |
| ATRA-1 | KRFKKFFKKLK-NH₂ | +8 |
| ATRA-2 | KRAKKFFKKPK-NH₂ | +8 |
| ATRA-1A | KRAKKFFKKLK-NH₂ | +8 |
| ATRA-1P | KRFKKFFKKPK-NH₂ | +8 |

*Based on analysis of the sequences using SSPro (http: //scratch.proteomics.ics.uci.edu), underlined residues are predicted to be in a helical conformation, those in normal text to be in a random coil conformation and those in italics to be in an extended conformation.

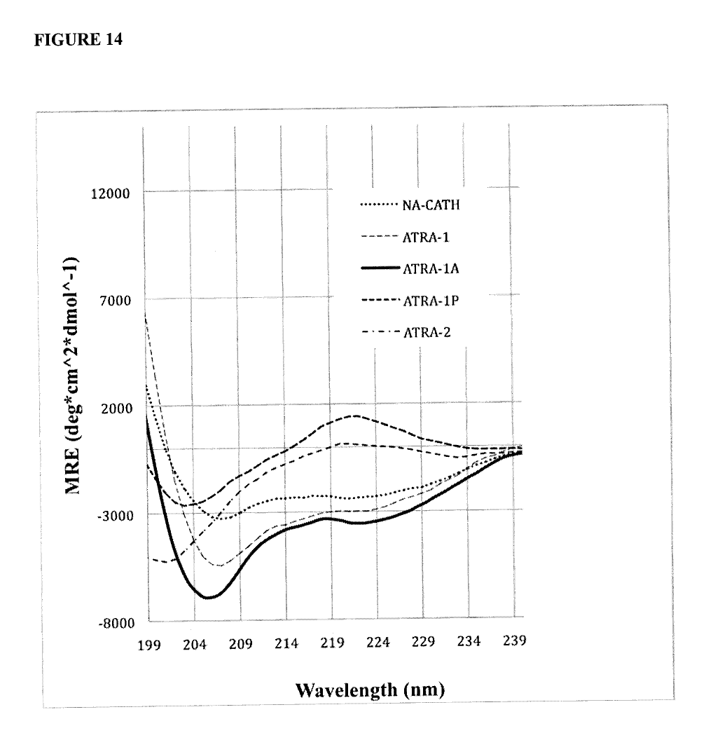

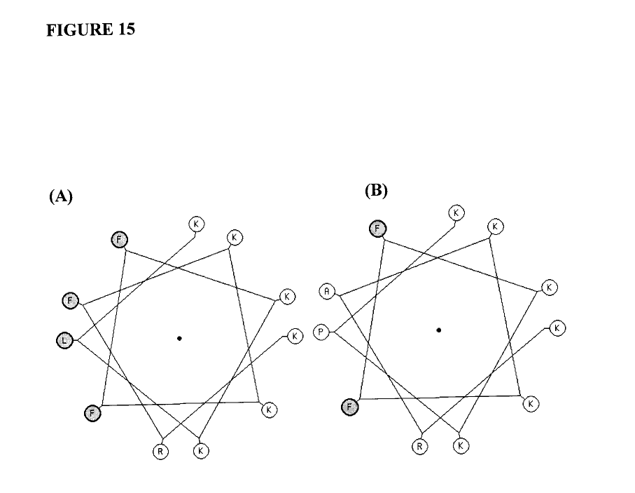

The structural properties of full-length NA-CATH and the truncated peptides were experimentally determined using circular dichroism (FIG. 14). While none of the peptides showed significant helical character in phosphate buffer (data not shown), full-length NA-CATH, ATRA-1 and ATRA-1A demonstrated varied degrees of α-helical structure in 90 mM SDS. When spectra were adjusted for peptide concentration and length, it was found that both ATRA-1 and ATRA-1A had greater per-residue helicity than did the full-length cathelicidin, with ATRA-1A being more helical than ATRA-1. Under these same conditions, the spectra for ATRA-2 and ATRA-1P were consistent with peptides with a random coil structure. These results were in agreement with the results from SSPro analysis of the sequences.

### Example 13

### Antibiotic Assay

Similar to Example 1 above, A. actinomycetemcomitans was incubated with various antibiotics independently. Specifically, A. actinomycetemcomitans was incubated with ciprofloxacin, gentamicin, metronidazole, metronidazole, azithromycin, ampicillin, and clindamycin.

Antibiotic assays were performed following previously published protocols with modification [8, 9]. The microorganisms were grown to mid-logarithmic phase appropriate broth and then diluted to 10E6 CFU/ml in Todd-Hewitt broth (for A. actinomycetemcomitans) and a pH 7.4.

Bacteria (50 μl) were incubated in the presence of different concentrations of peptide, from 10E-7 to 10E3 μg/ml. Assays were incubated at 37° C. for 3 h in 5% CO2, after which serial dilutions were prepared in 1× Dulbecco's Phosphate buffered Saline and then plated in triplicate onto appropriate agar plates. The plates were incubated at 37° C. overnight, and the colonies were counted after 24 hours for A. actinomycetemcomitans.

Bacterial survival at each peptide concentration was calculated based on the ratio of the number of colonies on the plates corresponding to the peptide concentration and the average number of colonies observed for assay cultures lacking peptide. The peptide concentration required to kill 50% of the viable bacteria in the assay cultures (EC50) was determined by plotting percent mortality as a function of the log of peptide concentration (log μg/ml) and fitting the data, using GraphPad Prism (GraphPad Software Inc., San Diego, Calif., USA), to Equation (1), which describes sigmoidal dose-response:

Y=Bottom+((Top-Bottom)/(1+10̂[(Log EC50−X)*Hill Slope] Equation 1

Where Y corresponds to bacterial mortality (%) at a given peptide concentration (ug/ml), with X being the logarithm of that concentration (log μg/ml). In the equation, “Top” and “Bottom” refer to the upper and lower boundaries, and were constrained to values <100% and >0%, respectively. For the purpose of graphing, samples that had no peptide are plotted at 10̂-9 μg/ml. EC50 values were determined by fitting the data from the antimicrobial assays to a standard sigmoidal dose-response curve (Eq. (1)). Errors were reported based on the standard deviation from the mean of the Log EC50 values.

As shown below in Table 5, ciprofloxacin was the most potent antibiotic.

TABLE 5
EC50's of A. actinomycetemcomitans Treated with Antibiotics

| Antibiotic | Molecular Weight | EC50 (μg/mL) | EC50 in μM |
|---|---|---|---|
| ciprofloxacin | 331.34 | 0.2829 | 0.8538 |
| gentamicin | 477.6 | 1.597 | 3.3438 |
| metronidazole | 171.15 | 19.48 | 113.8183 |
| amoxicillin | 365.4 | 29.56 | 80.897 |
| azithromycin | 748.98 | 34.4 | 45.929 |
| ampicillin | 349.4 | 74.99 | 214.625 |
| clindamycin | 461.44 | >250 | >541.78 |

As shown in Table 6, below SMAP-29 was the most potent peptide tested while ciprofloxacin was the most potent antibiotic tested.

TABLE 6
EC50's of All Antimicrobials Tested

| Antimicrobial | Molecular Weight | EC50 (μg/mL) | EC50 in μM |
|---|---|---|---|
| SMAP-29 | 3257 | 0.06386 | 0.0196 |
| K9CATH | 4512.25 | 0.5005 | 0.1109 |
| LL-37 Pentamide | 4523 | 0.7648 | 0.1691 |
| BMAP-28 | 3074.81 | 0.6616 | 0.2151 |
| Snake Full | 4175.22 | 1.653 | 0.3959 |
| Snake 1 | 1496 | 0.9263 | 0.6191 |
| Ciprofloxacin | 331.34 | 0.2829 | 0.8538 |
| Pmap-37 | 4364.98 | 5.465 | 1.252 |
| LL-37 | 4493.3 | 6.244 | 1.3896 |
| CAP-18 | 4433 | 7.049 | 1.5901 |
| Gentamicin | 477.6 | 1.597 | 3.3438 |
| Azithromycin | 748.98 | 34.4 | 45.929 |
| Amoxicillin | 365.4 | 29.56 | 80.897 |
| Snake 2 | 1395 | 158.3 | 113.4767 |
| Metronidazole | 171.15 | 19.48 | 113.8183 |
| Ampicillin | 349.4 | 74.99 | 214.625 |
| hBD-4 | 4366.1 | 100 | >22.9 |
| Peptide 4 | 1282.52 | 100 | >77.97 |
| Clindamycin | 461.44 | >250 | >541.78 |

### Example 14

### Biofilm Assay

Biofilm assays were performed by seeding a 96-well plate with an overnight culture of A. actinomycetemcomitans Y4 and incubating the plate for 24 hours at 37° C. and 5% CO₂. Liquid cultures were removed and the well were washed three times with 1×PBS. Biofilms were fixed by adding methanol to the wells for 15 minutes. Methanol was removed and crystal violet was added to the wells. Excess crystal violet was removed and the plates were washed carefully with distilled water to remove excess stain. Glacial acetic acid was then added to bring up the crystal violet stain and the intensity of the stain was quantified by measuring optical density (OD) with a microtiter plate reading spectrophotometer.

As shown n Table 7 below, A. actinomycetemcomitans biolfim can be detected and has an optical density almost 11× that of the negative control.

TABLE 7
A. actinomycetemcomitans Biofilm Production

| Sample | Optical Density (OD) |
|---|---|
| Todd Hewitt Broth (Negative Control) | 0.1018 |
| E. coli (Positive Control) | 0.376 |
| A. actinomycetemcomitans Y4 | 1.157 |

### Example 15

### Antimicrobial Activity of Cathelidins Against Francisella Bacterial and Mammalian Cells

F. novicida (F. tularensis novicida) (BEI NR-13) was obtained and grown in Tryptic Soy Broth supplemented with 0.1% Cysteine (TSB-C, 37° C., 24 h with shaking at 200 rpm), or on TSB-C agar or BD Chocolate Agar (GC II agar with IsoVitaleX™) plates. Cultures of F. novicida were grown up in one passage, stocks frozen in 20% glycerol and aliquots stored at −80° C. The CFU/ml was determined by growth on TSB-C agar. For bactericidal assays, frozen enumerated aliquots were thawed immediately prior to use. Overnight cultures were used for infection assays. Cell growth was monitored at O.D. 600 nm. The CFU/ml was determined with a standard curve of absorbance vs. CFU/ml. To each well of a multi-well sterile tissue culture plate bacteria were added at a MOI of 500 (bacteria: cells). Human A549 alveolar type II epithelial cells (ATCC CCL-185) were maintained following manufacturer's instructions.

### Bactericidal Assays

The antimicrobial activity of the NA-CATH (Genscript), the ATRA peptides (Genscript custom synthesis) and LL-37 (AnaSpec 61302) against F. novicida was determined as previously described. E. A. Papanastasiou, et al. APMIS 117 (2009) 492-9. Briefly, 1×10⁵ CFU per well of bacteria were incubated with different peptide concentrations in a 50-μl solution of Buffer Q containing 10 mM potassium phosphate and 1% TSB-C (3 h, 37° C.). Serial dilutions were then prepared in 1× Dulbecco's PBS and plated in triplicate on TSB-C plates, which were incubated (37° C., 48 hr) and CFUs counted. Bacterial survival at each peptide concentration was calculated based on the ratio of the number of colonies on each experimental plate and the average number of colonies observed for assay cultures lacking peptide. The peptide concentration required to kill 50% of the viable F. novicida in the assay cultures (EC50) was determined by plotting percent mortality as a function of the log of peptide concentration (log μg/ml) and fitting the data using GraphPad Prism 5 (GraphPad Software Inc., San Diego, Calif., USA). For the purpose of graphing, samples that had no peptide (0 μg/ml) are plotted at 10⁻⁹ μg/ml peptide. EC50 values were determined by fitting the data from the antimicrobial assays to a standard sigmoidal dose-response curve. Errors were reported based on the standard deviation from the mean of the log EC50 values. Student's T-test was used to determine whether points were statistically different.

We determined the time required for LL-37 and NA-CATH antimicrobial activity against F. novicida. 1×10⁵ CFU per well were incubated with 0.24 μg/ml of LL-37 and 1.54 μg/ml of NA-CATH in Buffer Q. The antimicrobial activity for each peptide was determined throughout the 3 hr incubation time. The appropriate dilutions of each well were plated in triplicate and the killing kinetics for the two peptides were determined.

### Broth Microdilution Assay

To determine the MIC, a broth microdilution assay was performed in a 96 well plate. 1×10⁵ CFU of bacteria per well were incubated with different peptide concentrations (250 μg/ml to 0.7 μg/ml, n=6) in a 200-μl solution of TSB-C (24 h, 37° C.). Bacterial growth was observed in all peptide wells, but not in negative control wells (peptide alone and broth alone). In addition, the EC50 was determined as described above, using TSB-C instead of in Buffer Q. Complete bacterial killing could not be achieved because the peptide concentration could not be made high enough. However, the EC50 could be estimated based on the percent killing at 250 and 125 μg/ml LL-37.

### Biofilm Production

Biofilm production was measured as previously described in M. W. Durham-Colleran et al., Microb Ecol (2009) with the following modifications. F. novicida (1×10⁵ CFU) in 250 μl final volume of fresh TSB-C and peptide beginning with 0.24 μg/ml LL-37 peptide (six wells per concentration) was incubated (48 h, 37° C.). Biofilm production was measured using the crystal violet stain technique M. W. Durham-Colleran et al., (2009).

Analysis of LL-37 Gene Expression by Real Time q(RT)-PCR

Quantitative real time RT-PCR analysis was performed in a MyiQ Single Color Real-Time PCR Detection System (BioRad Laboratories) as previously described S. Han et al., Biochem Biophys Res Commun 371 (2008) 670-4 with the following modifications. 1×10⁶ A549 cells were plated in a 6-well dish and serum starved overnight, then infected (500 MOI F. novicida) for 2 h, incubated with 50 μg/ml gentamicin for 1 h (removes extracellular bacteria), washed 3 times with PBS and replenished with 2 μg/ml gentamicin-containing medium for 24 hrs. Total RNA was isolated (RNAeasy Mini Kit, Qiagen) and 2 μg of total RNA were reverse-transcribed (Super Script™ III Reverse Transcriptase, Invitrogen). Template cDNA corresponding to 25 ng of RNA was added to 15 μl reaction: 0.2 μM each primer and 1× iQ Supermix (BioRad Laboratories). Samples were incubated in 96-well plate in the MyiQ Single Color Real-Time PCR Detection System. Initial denaturing: 95° C. for 3 min; 40 cycles consisting of 95° C. for 15 s, 60° C. for 15 s and 72° C. for 20 s. SYBR Green (BioRad Laboratories) fluorescence was detected at 72° C. at the end of each cycle. Melting curve profiles were produced (cooling the sample to 60° C. for 1 min and then heating slowly at 0.5° C./s up to 95° C. with continuous measurement of fluorescence to confirm amplification of specific transcripts. LL-37 primer sequence 5′-CTAGAGGGAGGCAGACATGG-3′ forward and 5′-AGGAGGCGGTAAGGTTAGC-3′ reverse were obtained from RealTimePrimers, LLC (Elkins Park, Pa.), resulting in 201 base pair fragment. Relative LL-37 transcript levels were corrected by normalization based on the 18S transcript levels. Amplification products were verified by electrophoresis on a 2% agarose gel and visualized by ethidium bromide staining. For statistical analysis, determinations were performed in triplicates. Error bars represent the standard deviation from the mean of the experimental set. Where required, t-tests were performed to compare sample sets and the p-value was reported.

### B. Results

### LL-37 and NA-CATH Exert Direct Antimicrobial Effects on Francisella

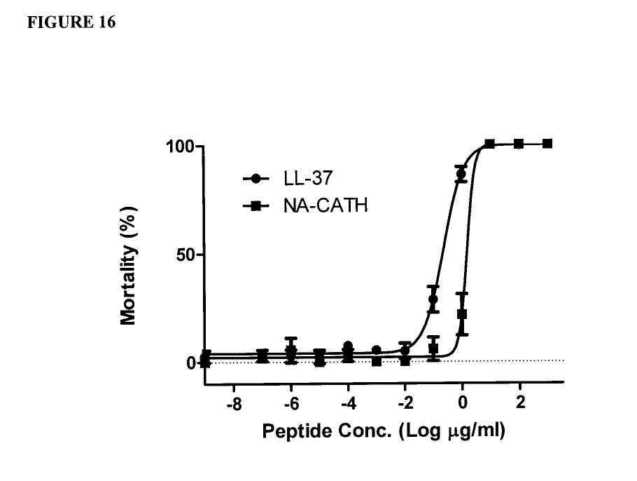

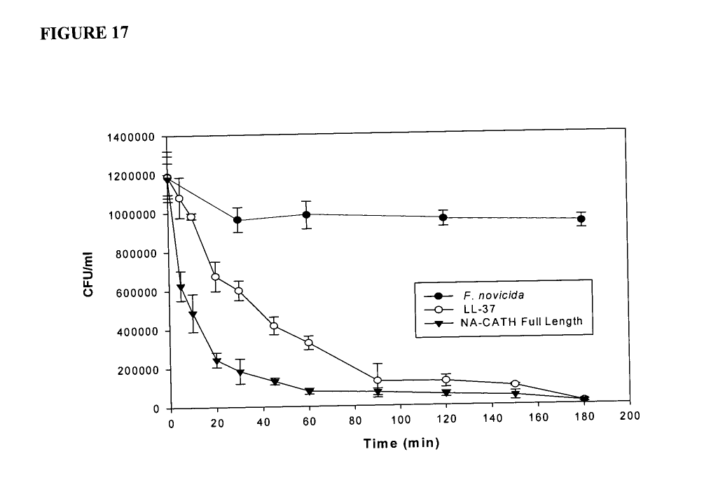

In this study, the antimicrobial effectiveness of various peptides including NA-CATH (Table 1) and the smaller ATRA motif-based peptides were tested against F. novicida, and their performances were compared to that of LL-37. We determined the EC50 value for LL-37 to be 0.24 μg/ml in Buffer Q and for NA-CATH to be 1.54 μg/ml (FIG. 16). Thus, LL-37 is more effective against F. novicida than NA-CATH. This is in contrast to E. coli and A. actinomycetemcomitans against which the NA-CATH is more effective, as has also been shown for OH-CATH. H. Zhao et al., Identification and characterization of novel reptile cathelicidins from elapid snakes. Peptides 29 (2008) 1685-91. To determine the time required for antimicrobial activity of the peptides, killing kinetics for LL-37 and NA-CATH were determined against F. novicida. The antimicrobial activity for each peptide was determined over 3 h. We found that NA-CATH kills Francisella more rapidly than the LL37 (FIG. 17), but by 90 min both peptides had killed most of the bacteria.

### ATRA Peptides Exert Direct Antimicrobial Effects on Francisella

Francisella was also subjected to treatment with two shorter length synthetic peptides ATRA-1 and ATRA-2, which represent the ATRA-repeated motif of NA-CATH. The two peptides differ by only two residues at the third (F/A) and tenth (L/P) position. We predicted that this difference in peptide sequence could affect the antimicrobial activity of those peptides. The side chain of alanine is much smaller than phenylalanine, which may affect the hydrophobic face of the peptide. Proline tends to destabilize and disrupt the helical structure of peptides. This may impact the ability of the ATRA-2 to achieve a helical conformation when interacting with membranes. The EC50 values of ATRA-1 and ATRA-2 were determined to be 8.96 μg/ml and 147.9 μg/ml respectively (Table 2). These two peptides have the same net charge of +8, highly similar sequence and the same length (11 residues). However, the potency of ATRA-1 against F. novicida is ˜20-fold that of ATRA-2, indicating that the sequence differences may influence activity of the peptides.

### Contribution of Position 3 and 10 to ATRA Peptide Activity

To understand the contribution of positions three and ten on the peptide activity, we synthesized two new peptides (ATRA-1A and ATRA-1P) (Table 1). The phenylalanine in the third position of ATRA-1 was replaced by an alanine for ATRA-1A, and the leucine at the tenth position was replaced by a proline for ATRA-1P. The EC50 values of ATRA-1A and ATRA-1P were determined to be 11.34 μg/ml and 141.3 μg/ml respectively (Table 2). These vastly different results may reflect the contribution of the proline in altering the alpha-helical nature of the peptide in ATRA-1P which we have shown to be significantly different than ATRA-1, thus interfering with the peptide's ability to exert a strong antimicrobial activity against F. novicida. In another project, we have determined that none of these ATRA peptides induced hemolysis even at 100 μg/ml, which is much higher than the effective concentration of the active peptides (0.2 to 11.34 μg/ml).

TABLE 1
Sequences of Antimicrobial Peptides

| | | Net |
|---|---|---|
| Peptide | Sequence | Charge |
| NA-CATH | KRFKKFFKKLKNSVKKRAKKFFKKPKVIGVTFPF | +15 |
| | (ATRA-1) (ATRA-2) | |
| ATRA-1 | KRFKKFFKKLK-NH2 | +8 |
| ATRA-2 | KRAKKFFKKPK-NH2 | +8 |
| ATRA-1A | KRAKKFFKKLK-NH2 | +8 |
| ATRA-1P | KRFKKFFKKPK-NH2 | +8 |
| LL-37 | LLGDFFRKSKEKIGKEFKRIVQRIKDFLRNLVPR | +8 |
| | TES | |

Table 1. Sequences of Antimicrobial Peptides. Bold indicates the repeated motifs of the NA-CATH peptide. Underlined sequences indicated indicate positions three and ten of the peptide.

TABLE 2
EC50s of Antimicrobial Peptides against F. novicida.

| Antimicrobial | Molecular | EC50 | Fold | EC50 | Fold |
|---|---|---|---|---|---|
| Peptide | Weight (g/mol) | μg/ml | Change | μM | Change |
| NA-CATH | 4175.22 | 1.54 | 1.0 | 0.37 | 1.0 |
| ATRA-1 | 1496.94 | 8.95 | 5.8 | 5.98 | 16.2 |
| ATRA-2 | 1404.80 | 147.90 | 97.7 | 107.0 | 290 |
| ATRA-1A | 1420.84 | 11.34 | 7.4 | 7.98 | 21.7 |
| ATRA-1P | 1480.89 | 141.30 | 91.9 | 95.42 | 259 |
| LL-37 | 4493.3 | 0.24 | 0.2 | 0.05 | 0.1 |

EC50 is expressed both as μg/ml and as μM, accounting for the molecular weight of each peptide.

### LL-37 Inhibits Francisella Biofilm Formation at Sub-Antimicrobial Concentrations.

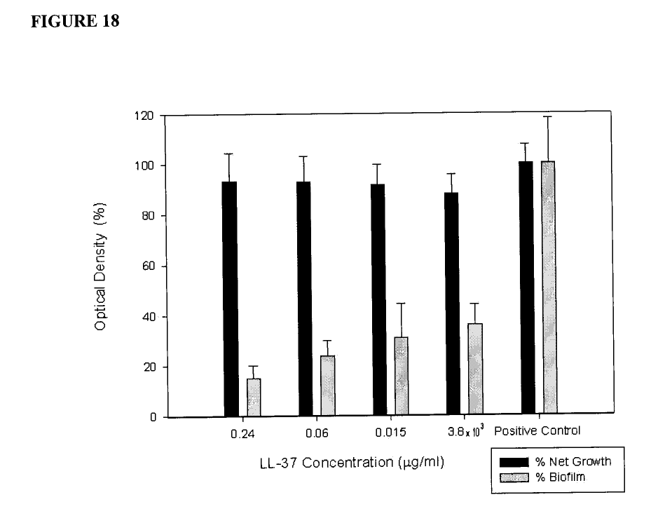

It has been described that the LL-37 cathelicidin can inhibit the formation of P. aeruginosa biofilms at a concentration of 0.05 μg/ml, which is well below that required to kill or inhibit growth in broth microdilution assays (MIC=64 μg/ml) J. Overhage, Infect Immun 76 (2008) 4176-82. For F. novicida, the MIC of LL-37 is greater than 250 μg/ml in broth microdilution assay (data not shown) and the estimated EC50 in broth is approximately 15 μg/ml (data not shown). We have recently reported the ability of F. novicida to form in vitro biofilms M. W. Durham-Colleran et al., (2009). Because of its role in airway defense, we decided to test the capacity of LL-37 to inhibit biofilm formation by F. novicida. We incubated various concentrations of LL-37 with F. novicida in a biofilm experiment in 100% bacterial media (TSB-C) under conditions that allow biofilm formation M. W. Durham-Colleran et al., (2009). FIG. 18 demonstrates that the growth levels observed in this study were similar to that of untreated F. novicida, even in the presence of as much as 0.24 μg/ml LL37. This indicates that there is no inhibition of growth at this peptide concentration. When we measured the cultures for biofilm production, significant inhibition of biofilm formation by LL-37 was observed even at a concentration as low as 3.8 ng/ml.

Human Cathelicidin LL-37 mRNA Expression in A549 Cells Infected with Francisella.

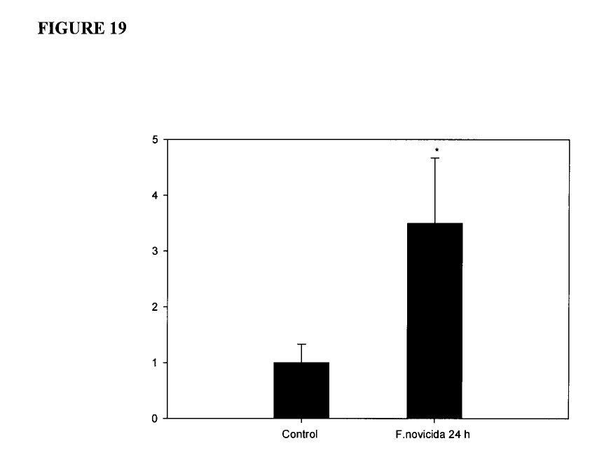

Antimicrobial peptides are a major player in local immunity, especially at mucosal and epithelial surfaces. It is of interest to determine the induction of LL-37 in infected A549 cells with Francisella. A549 cells are a human alveolar epithelial cell line that we use as a model of aerosol exposure and infection to tularemia. Accordingly, qRT-PCR was used to determine the total amount of LL-37 mRNA present in A549 cells following F. novicida infection. We determined that LL-37 mRNA levels were elevated 3.5 fold on average relative to the mRNA levels in uninfected control cells (p-value: 0.024) (FIG. 19). This is the first report of LL-37 expression being induced in A549 cells as a consequence of Francisella stimulus or infection.

### REFERENCES

(1) Wilson M, Henderson, B. Virulence factors of A. actinomycetemcomitans revenant to the pathogenesis of inflammatory periodontal diseases. FEMS 1995:17:365-379.
(2) Nowotny A, Behling U H, Hammond B, et al. Release of toxic microvesicles by Actinobacillus actinomycetemcomitans. Infect Immun 1982:37:151-154.
(3) Kaplan J B, Perry M B, MacLean L L, Furgang D, Wilson M E, Fine D H. Structural and genetic analyses of 0 polysaccharide from Actinobacillus actinomycetemcomitans serotype f. Infect Immun 2001:69:5375-5384.
(4) Rosan B, Slots J, Lamont R J, Listgarten M A, Nelson G M. Actinobacillus actinomycetemcomitans fimbriae. Oral Microbiol Immunol 1988:3:58-63.
(5) Inouye T, Ohta H, Kokeguchi S, Fukui K, Kato K. Colonial variation and fimbriation of Actinobacillus actinomycetemcomitans. FEMS microbiology letters 1990: 57: 13-17.
(6) Kaplan J B, Schreiner H C, Furgang D, Fine D H. Population structure and genetic diversity of Actinobacillus actinomycetemcomitans strains isolated from localized juvenile periodontitis patients. Journal of clinical microbiology 2002:40:1181-1187.
(7) Slots J, Reynolds H S, Genco R J. Actinobacillus actinomycetemcomitans in human periodontal disease: a cross-sectional microbiological investigation. Infect Immun 1980:29:1013-1020.
(8) Armitage G C. Development of a classification system for periodontal diseases and conditions. Annals of periodontology/the American Academy of Periodontology 1999:4:1-6.
(9) Tonetti M S, Mombelli A. Early-onset periodontitis. Annals of periodontology/the American Academy of Periodontology 1999:4:39-53.
(10) Wisner-Lynch L A, Giannobile W V. Current concepts in juvenile periodontitis. Current opinion in periodontology 1993: 28-42.
(11) Henderson B, Wilson, M., Sharp, L., Ward, J. Actinobacillus actinomycetemcomitans. J Med Micro 2002:51:1013-1020.
(12) Page M I, King E O. Infection due to Actinobacillus actinomycetemcomitans and Haemophilus aphrophilus. The New England journal of medicine 1966:275:181-188.
(13) Muhle I, Rau J, Ruskin J. Vertebral osteomyelitis due to Actinobacillus actinomycetemcomitans. Jama 1979:241:1824-1825.
(14) Blair T P, Seibel J, Jr., Oldfield E, Berg S W, Karney W, Baker W P. Endocarditis caused by Actinobacillus actinomycetemcomitans. Southern medical journal 1982: 75: 559-561.
(15) Thoden van Velzen S K, Abraham-Inpijn, L., Moorer, W. R. Plaque and systemic disease: a reapprasial of the focal infection concept. J Clin Periodontology 1984: 11: 209-220.
(16) Marsh P D. Dental plaque: biological significance of a biofilm and community life-style. Journal of clinical periodontology 2005: 32 Suppl 6: 7-15.
(17) Marsh P D. Dental plaque as a biofilm and a microbial community—implications for health and disease. BMC oral health 2006: 6 Suppl 1: S14.
(18) Paju S, Scannapieco F A. Oral biofilms, periodontitis, and pulmonary infections. Oral diseases 2007:13:508-512.
(19) Bowden G H, Hamilton I R. Survival of oral bacteria. Crit. Rev Oral Biol Med 1998: 9: 54-85.
(20) Haake S. Microbiology of Dental Plaque. In.
(21) Wagner V E, Frelinger J G, Barth R K, Iglewski B H. Quorum sensing: dynamic response of Pseudomonas aeruginosa to external signals. Trends in microbiology 2006:14:55-58.
(22) Frias J, Olle E, Alsina M. Periodontal pathogens produce quorum sensing signal molecules. Infect Immun 2001:69:3431-3434.
(23) Chung W O, Park Y, Lamont R J, McNab R, Barbieri B, Demuth D R. Signaling system in Porphyromonas gingivalis based on a LuxS protein. Journal of bacteriology 2001:183:3903-3909.
(24) Fong K P, Chung W O, Lamont R J, Demuth D R. Intra- and interspecies regulation of gene expression by Actinobacillus actinomycetemcomitans LuxS. Infect Immun 2001:69:7625-7634.
(25) Sliepen I, Van Essche M, Quirynen M, Teughels W. Effect of mouthrinses on Aggregatibacter actinomycetemcomitans biofilms in a hydrodynamic model. Clinical oral investigations 2009.
(26) Stepanovic S, Vukovic D, Dakic I, Savic B, Svabic-Vlahovic M. A modified microtiter-plate test for quantification of staphylococcal biofilm formation. Journal of microbiological methods 2000:40:175-179.
(27) O'Toole G A, Kolter R. Flagellar and twitching motility are necessary for Pseudomonas aeruginosa biofilm development. Molecular microbiology 1998: 30: 295-304.
(28) O'Toole G A, Kolter R. Initiation of biofilm formation in Pseudomonas fluorescens WCS365 proceeds via multiple, convergent signalling pathways: a genetic analysis. Molecular microbiology 1998:28:449-461.
(29) Beloin C, Roux A, Ghigo J M. Escherichia coli biofilms. Current topics in microbiology and immunology 2008:322:249-289.
(30) Tang Y Q, Yuan J, Miller C J, Selsted M E. Isolation, characterization, cDNA cloning, and antimicrobial properties of two distinct subfamilies of alpha-defensins from rhesus macaque leukocytes. Infect Immun 1999:67:6139-6144.
(31) Han S, Bishop B M, van Hoek M L. Antimicrobial activity of human beta-defensins and induction by Francisella. Biochem Biophys Res Commun 2008:371:670-674.
(32) Zasloff M. Antimicrobial peptides of multicellular organisms. Nature 2002: 415: 389-395.
(33) Jenssen H, Hamill P, Hancock R E. Peptide antimicrobial agents. Clinical microbiology reviews 2006:19:491-511.
(34) Weinberg A, Krisanaprakornkit S, Dale B A. Epithelial antimicrobial peptides: review and significance for oral applications. Crit. Rev Oral Biol Med 1998:9:399-414.
(35) Dale B A, Fredericks L P. Antimicrobial peptides in the oral environment: expression and function in health and disease. Current issues in molecular biology 2005: 7: 119-133.
(36) Tomasinsig L, Zanetti M. The cathelicidins—structure, function and evolution. Current protein & peptide science 2005:6:23-34.
(37) Oren Z, Lerman J C, Gudmundsson G H, Agerberth B, Shai Y. Structure and organization of the human antimicrobial peptide LL-37 in phospholipid membranes: relevance to the molecular basis for its non-cell-selective activity. The Biochemical journal 1999: 341 (Pt 3): 501-513.
(38) Ganz T. Defensins: antimicrobial peptides of vertebrates. Comptes rendus biologies 2004:327:539-549.
(39) Verma C, Seebah S, Low S M, et al. Defensins: antimicrobial peptides for therapeutic development. Biotechnology journal 2007:2:1353-1359.
(40) Yang C, Boone L, Nguyen T X, et al. theta-Defensin pseudogenes in HIV-1-exposed, persistently seronegative female sex-workers from Thailand. Infect Genet Evol 2005: 5: 11-15.
(41) Schutte B C, Mitros J P, Bartlett J A, et al. Discovery of five conserved beta-defensin gene clusters using a computational search strategy. Proceedings of the National Academy of Sciences of the United States of America 2002:99:2129-2133.
(42) Larrick J W, Hirata M, Shimomoura Y, et al. Antimicrobial activity of rabbit CAP18-derived peptides. Antimicrobial agents and chemotherapy 1993:37:2534-2539.
(43) Larrick J W, Hirata M, Shimomoura Y, et al. Rabbit CAP18 derived peptides inhibit gram negative and gram positive bacteria. Progress in clinical and biological research 1994:388:125-135.
(44) Guthmiller J M, Vargas K G, Srikantha R, et al. Susceptibilities of oral bacteria and yeast to mammalian cathelicidins. Antimicrobial agents and chemotherapy 2001: 45: 3216-3219.
(45) Sang Y, Teresa Ortega M, Rune K, et al. Canine cathelicidin (K9CATH): gene cloning, expression, and biochemical activity of a novel pro-myeloid antimicrobial peptide. Developmental and comparative immunology 2007:31:1278-1296.
(46) Skerlavaj B, Gennaro R, Bagella L, Merluzzi L, Risso A, Zanetti M. Biological characterization of two novel cathelicidin-derived peptides and identification of structural requirements for their antimicrobial and cell lytic activities. J Biol Chem 1996:271:28375-28381.
(47) Risso A, Braidot E, Sordano M C, et al. BMAP-28, an antibiotic peptide of innate immunity, induces cell death through opening of the mitochondrial permeability transition pore. Molecular and cellular biology 2002:22:1926-1935.
(48) Gennaro R, Zanetti M. Structural features and biological activities of the cathelicidin-derived antimicrobial peptides. Biopolymers 2000:55:31-49.
(49) Benincasa M, Skerlavaj B, Gennaro R, Pellegrini A, Zanetti M. In vitro and in vivo antimicrobial activity of two alpha-helical cathelicidin peptides and of their synthetic analogs. Peptides 2003:24:1723-1731.
(50) Brogden K A, Nordholm G, Ackermann M. Antimicrobial activity of cathelicidins BMAP28, SMAP28, SMAP29, and PMAP23 against Pasteurella multocida is more broad-spectrum than host species specific. Veterinary microbiology 2007:119:76-81.
(51) Skerlavaj B, Benincasa M, Risso A, Zanetti M, Gennaro R. SMAP-29: a potent antibacterial and antifungal peptide from sheep leukocytes. FEBS letters 1999: 463: 58-62.
(52) Mahoney M M, Lee A Y, Brezinski-Caliguri D J, Huttner K M. Molecular analysis of the sheep cathelin family reveals a novel antimicrobial peptide. FEBS letters 1995: 377: 519-522.
(53) Tack B F, Sawai M V, Kearney W R, et al. SMAP-29 has two LPS-binding sites and a central hinge. European journal of biochemistry/FEBS 2002:269:1181-1189.
(54) Joly S, Maze C, McCray P B, Jr., Guthmiller J M. Human beta-defensins 2 and 3 demonstrate strain-selective activity against oral microorganisms. Journal of clinical microbiology 2004:42:1024-1029.
(55) Franzman M R, Burnell K K, Dehkordi-Vakil F H, Guthmiller J M, Dawson D V, Brogden K A. Targeted antimicrobial activity of a specific IgG-SMAP28 conjugate against Porphyromonas gingivalis in a mixed culture. International journal of antimicrobial agents 2009:33:14-20.
(56) Zhao H, Gan T X, Liu X D, et al. Identification and characterization of novel reptile cathelicidins from elapid snakes. Peptides 2008:29:1685-1691.
(57) Tossi A, Scocchi M, Zanetti M, Storici P, Gennaro R. PMAP-37, a novel antibacterial peptide from pig myeloid cells. cDNA cloning, chemical synthesis and activity. European journal of biochemistry/FEBS1995:228:941-946.
(58) Sang Y, Blecha F. Porcine host defense peptides: expanding repertoire and functions. Developmental and comparative immunology 2009:33:334-343.
(59) Nijnik A, Hancock R E. The roles of cathelicidin LL-37 in immune defenses and novel clinical applications. Current opinion in hematology 2009:16:41-47.
(60) Di Nardo A, Braff M H, Taylor K R, et al. Cathelicidin antimicrobial peptides block dendritic cell TLR4 activation and allergic contact sensitization. J Immunol 2007: 178: 1829-1834.
(61) Lau Y E, Rozek A, Scott M G, Goosney D L, Davidson D J, Hancock R E. Interaction and cellular localization of the human host defense peptide LL-37 with lung epithelial cells. Infect Immun 2005:73:583-591.
(62) De Smet K, Contreras R. Human antimicrobial peptides: defensins, cathelicidins and histatins. Biotechnology letters 2005:27:1337-1347.
(63) Schaller-Bals S, Schulze A, Bals R. Increased levels of antimicrobial peptides in tracheal aspirates of newborn infants during infection. American journal of respiratory and critical care medicine 2002:165:992-995.
(64) Bals R, Weiner D J, Meegalla R L, Wilson J M. Transfer of a cathelicidin peptide antibiotic gene restores bacterial killing in a cystic fibrosis xenograft model. J Clin Invest 1999:103:1113-1117.
(65) Starner T D, Agerberth B, Gudmundsson G H, McCray P B, Jr. Expression and activity of beta-defensins and LL-37 in the developing human lung. J Immunol 2005: 174: 1608-1615.
(66) Ong P Y, Ohtake T, Brandt C, et al. Endogenous antimicrobial peptides and skin infections in atopic dermatitis. The New England journal of medicine 2002: 347: 1151-1160.
(67) Zhao C, Nguyen T, Boo L M, et al. RL-37, an alpha-helical antimicrobial peptide of the rhesus monkey. Antimicrobial agents and chemotherapy 2001:45:2695-2702.
(68) Tanaka D, Miyasaki K T, Lehrer R I. Sensitivity of Actinobacillus actinomycetemcomitans and Capnocytophaga spp. to the bactericidal action of LL-37: a cathelicidin found in human leukocytes and epithelium. Oral Microbiol Immunol 2000:15:226-231.
(69) Ouhara K, Komatsuzawa H, Yamada S, et al. Susceptibilities of periodontopathogenic and cariogenic bacteria to antibacterial peptides, {beta}-defensins and LL37, produced by human epithelial cells. The Journal of antimicrobial chemotherapy 2005:55:888-896.
(70) Altman H, Steinberg D, Porat Y, et al. In vitro assessment of antimicrobial peptides as potential agents against several oral bacteria. The Journal of antimicrobial chemotherapy 2006:58:198-201.

1. (canceled)

2. An isolated peptide conferring antimicrobial activity against a gram-negative bacterium wherein said peptide is K9CATH, BMAP-28, ATRA-1, ATRA-2, ATRA-1A, ATRA-1P, or PMAP-37.

3. The isolated peptide of claim 2, wherein said gram-negative bacterium is A. actinomycetemcomitans, F. tularensis, or E. coli.

4.-6. (canceled)

7. A vector comprising a sequence encoding at least one of ATRA-1, ATRA-2, ATRA-1A, and ATRA-1P.

8.-10. (canceled)

11. A method for sterilizing a surface against a gram-negative bacterium, comprising contacting said surface with at least one of K9CATH, BMAP-28, ATRA-1, ATRA-2, ATRA-1A, ATRA-1P, or PMAP-37.

12. The method of claim 10, wherein said gram-negative bacterium is A. actinomycetemcomitans, F. tularensis, or E. coli.

13.-14. (canceled)

15. A mouthwash comprising at least one of ATRA-1, ATRA-2, ATRA-1A, and ATRA-1P.


SEQUENCE LISTING (12 sequences)

SEQ ID NO: 1 - protein, 8 aa, Ovis sp.
RGLRRLGR

SEQ ID NO: 2 - protein, 8 aa, Ovis sp.
VLRIIRIA

SEQ ID NO: 3 - protein, 11 aa, Naja atra
  MOD_RES (3)..(3): Phe or Ala
KRXKKFFKKXK

SEQ ID NO: 4 - protein, 11 aa, Naja atra
KRFKKFFKKLK

SEQ ID NO: 5 - protein, 11 aa, Naja atra
KRAKKFFKKPK

SEQ ID NO: 6 - protein, 34 aa, Naja atra
KRFKKFFKKLKNSVKKRAKKFFKKPKVIGVTFPF

SEQ ID NO: 7 - protein, 11 aa, Artificial Sequence
  Description of Artificial Sequence Synthetic peptide
KRAKKFFKKLK

SEQ ID NO: 8 - protein, 11 aa, Artificial Sequence
  Description of Artificial Sequence Synthetic peptide
KRFKKFFKKPK

SEQ ID NO: 9 - protein, 37 aa, Homo sapiens
LLGDFFRKSKEKIGKEFKRIVQRIKDFLRNLVPRTES

SEQ ID NO: 10 - protein, 10 aa, Unknown
  Description of Unknown Bovine cathelicidin peptide
KKFKKLFKKL

SEQ ID NO: 11 - DNA, 20 bp, Artificial Sequence
  Description of Artificial Sequence Synthetic primer
ctagagggaggcagacatgg

SEQ ID NO: 12 - DNA, 19 bp, Artificial Sequence
  Description of Artificial Sequence Synthetic primer
aggaggcggtaaggttagc